(19) United States
(12) Patent Application Publication
(10) Pub. No.: US 2026/0321383 A1
(43) Pub. Date: Oct. 15, 2026

(54) LEVERAGING CONTINUOUS GLUCOSE, KETONE, LACTATE MONITORING DATA AND DIETARY INFORMATION FROM FOOD APPLICATIONS TO INFORM GLP-1 RA THERAPY

(71) Applicant: ABBOTT DIABETES CARE INC., Alameda, CA (US)

(72) Inventors: Aparajita Bhattacharya, Dublin, CA (US); Amruta Pai, Fremont, CA (US); Anila Bindal, Chicago, IL (US); Gary Alan Hayter, Oakland, CA (US); Byron Paul Olson, II, Sioux Falls, SD (US); Timothy Christian Dunn, San Francisco, CA (US); Samia Rahman, Alameda, CA (US)

(21) Appl. No.: 19/637,888

(22) Filed: Apr. 2, 2026

Related U.S. Application Data
(60) Provisional application No. 63/788,340, filed on Apr. 14, 2025.
(60) Provisional application No. 63/877,511, filed on Sep. 8, 2025.

(51) Int. Cl. G16H 20/17; G16H 20/60; G16H 40/67; G16H 50/20; G16H 50/30

(52) U.S. Cl. CPC G16H 20/17; G16H 20/60; G16H 40/67; G16H 50/20; G16H 50/30

(57) ABSTRACT

A service treats a user by recommending individualized insulin dose reductions prior to initiation of a therapy involving using a glucagon-like peptide-1 receptor agonist (GLP-1 RA) treatment, where the GLP-1 RA treatment is designed to treat a physiological condition of the user, and where a wearable monitor measures a physiological level of the user. The measured physiological level is used to facilitate the GLP-1 RA treatment.

### CROSS-REFERENCE TO RELATED APPLICATIONS

This application claims the benefit of and priority to United States Provisional Patent Application Serial No. 63/788,340 filed on Apr. 14, 2025 and entitled “USING MACHINE LEARNING TO MODIFY GLP-1 TREATMENTS BASED ON CGM DATA,” which application is expressly incorporated herein by reference in its entirety.

This application also claims the benefit of and priority to United States Provisional Patent Application Serial No. 63/877,511 filed on Sep. 8, 2025 and entitled “LEVERAGING CONTINUOUS GLUCOSE, KETONE, LACTATE MONITORING DATA AND DIETARY INFORMATION FROM FOOD APPLICATIONS TO INFORM GLP-1RA THERAPY,” which application is expressly incorporated herein by reference in its entirety.

### BACKGROUND

A continuous glucose monitor (CGM) is a type of analyte sensing device that tracks a user’s blood glucose levels. As used herein, the term “analyte” refers to a substance that is the subject of an analysis. For instance, the CGM tracks glucose levels, and glucose is considered the analyte.

A “glucose level” is also commonly referred to as a “blood sugar” level. The CGM may include a very small sensor that enters or permeates at least partially through the user’s skin. Typically, the CGM will acquire a new reading on a periodic basis, such as once every select number of minutes. The CGM includes a wireless transmitter that then sends the reading to a receiving device, such as a smartphone.

The detection and/or monitoring of analyte levels, such as glucose, ketones, lactate, alcohol, oxygen, hemoglobin A1C, or the like, can be vitally important to the health of an individual having diabetes. Patients suffering from diabetes mellitus can experience complications including loss of consciousness, cardiovascular disease, retinopathy, neuropathy, and nephropathy. Diabetics are generally required to monitor their glucose levels to ensure that they are being maintained within a clinically safe range and may also use this information to determine if and/or when insulin is needed to reduce glucose levels in their bodies, or when additional glucose is needed to raise the level of glucose in their bodies.

Growing clinical data demonstrates a strong correlation between the frequency of glucose monitoring and glycemic control. Despite such correlation, many individuals diagnosed with a diabetic condition do not monitor their glucose levels as frequently as they should due to a combination of factors, including convenience, testing discretion, pain associated with glucose testing, and cost.

To increase patient adherence to a plan of frequent glucose monitoring, in vivo analyte monitoring systems can be utilized (e.g., such as the above described CGM), in which a sensor control device may be worn on the body of an individual who requires analyte monitoring. To increase comfort and convenience for the individual, the sensor control device may have a small form-factor and can be assembled and applied by the individual with a sensor “applicator.”

The application process involves inserting at least a portion of a sensor, which senses a user’s analyte level in a bodily fluid located in a layer of the human body, using an applicator or insertion mechanism, such that the sensor comes in to contact with the bodily fluid. The sensor control device may also be configured to transmit analyte data to another device, from which the individual or her health care provider (“HCP”) can review the data and make therapy decisions.

Natural GLP-1 (Glucagon-like peptide-1) is a type of natural hormone in a human’s body. This hormone helps regulate a person’s blood sugar, digestion, and appetite. A “GLP-1 receptor agonist” (GLP-1 RA) is a type of pharmaceutical that mimics the natural GLP-1. Throughout this disclosure, the shorthand terms “GLP-1 RA” and “GLP-1” will be used to refer to the pharmaceutical while the phrase “natural GLP-1” will be used to refer to the naturally occurring human hormone. GLP-1s have been approved to treat type 2 diabetes. Recently, GLP-1s have also been used to help combat obesity by helping users lower their blood sugar levels and curb their appetites.

The subject matter claimed herein is not limited to embodiments that solve any disadvantages or that operate only in environments such as those described above. Rather, this background is only provided to illustrate one exemplary technology area where some embodiments described herein may be practiced.

### BRIEF DESCRIPTION OF THE DRAWINGS

In order to describe the manner in which the above-recited and other advantages and features can be obtained, a more particular description of the subject matter briefly described above will be rendered by reference to specific embodiments which are illustrated in the appended drawings. Understanding that these drawings depict only typical embodiments and are not therefore to be considered to be limiting in scope, embodiments will be described and explained with additional specificity and detail through the use of the accompanying drawings in which:

FIG. 1 illustrates an example architecture in which sensor data is being acquired and analyzed.

FIG. 2 illustrates an example of an analyte sensor in the form of a continuous glucose monitor.

FIG. 3 illustrates a chart that is plotting data obtained from the analyte sensor.

FIG. 4 illustrates a chart showing how data is intermittently or sparsely being obtained from one or more analyte sensors.

FIGS. 5A and 5B illustrate different time periods during which data is obtained.

FIG. 6A illustrates various different types of input data, and FIG. 6B shows an example of a GPI report.

FIGS. 7, 8, 9 and 10, illustrate various different user interfaces of an application.

FIGS. 11A and 11B illustrate flowcharts of example methods for generating a recommendation regarding a GLP-1 dosage.

FIGS. 12A and 12B illustrate an example graph representative of various characteristics and example flowchart.

FIG. 13 illustrates an example computer system that can be configured to perform any of the disclosed operations.

### DETAILED DESCRIPTION

The disclosed embodiments are directed to systems, methods, and devices (as well as computer programs and computer readable mediums) for using wearable monitor data (e.g. CGM sensor data) to help GLP-1 patients control their physiological levels, particularly their glucose levels. Beneficially, the embodiments can use CGM sensors continuously or in an intermittent manner to determine (i) when to escalate a certain therapy (e.g., a diabetes therapy), (ii) whether a GLP-1 is working or to predict whether the GLP-1 will likely work for a given patient (e.g., for successful weight loss or diabetes management (e.g., lowering A1C and/or GMI levels below a threshold)), (iii) derive insights from CGM data (e.g., whether or when to skip meals), and/or (iv) determine when and how to titrate a GLP-1 dose. Use of the disclosed principles can beneficially increase the number of CGM users. Additionally, the embodiments can beneficially introduce non-diabetic users to CGM sensors, can introduce finger-stick diabetic patients to CGMs, and can support at least intermittent use of CGM sensors for diabetic patients on GLP-1s. The disclosed embodiments can also be implemented with population based models. These population based models can be used to predict success. One possible outcome can be to recommend not starting GLP-1s and/or recommending the initial dose based on the population model or a personalized model (e.g., in a scenario where the user was previously on a GLP-1). It should also be noted how successful diabetes management can include less glucose variability, higher time in range, and/or lower GMI/A1C levels (e.g., below a threshold value such as 7%).

In some implementations, the population-based model may be generated using aggregated data obtained from a plurality of users, where the aggregated data includes one or more of physiological measurements, therapy information, and outcome data associated with prior users. The population-based model may be trained to identify relationships between input features (e.g., analyte levels, medication regimens, or dietary patterns) and corresponding outcomes (e.g., glycemic response, weight change, or therapy adherence). In operation, the population-based model may be applied to a specific user by providing that user’s input data to generate an initial prediction or recommendation. In some scenarios, the model may then be further adapted using data specific to the user, such as historical sensor data or prior responses to therapy, to produce a more personalized model. This adaptation may occur by adjusting model parameters, modifying feature weightings, or selecting among multiple candidate models based on similarity between the user and subsets of the population data. In this way, the disclosed embodiments support both population-level generalization and user-specific personalization using concrete data sources and processing steps.

In some implementations, the system may iteratively update its internal representations based on observed outcomes following prior recommendations. For example, if a recommendation results in a particular physiological response, that response may be incorporated into subsequent model updates, enabling the system to refine future recommendations for the same user.

In some implementations, the system may generate recommendations without relying on population-derived expected outcomes or thresholds. Instead, the model may prioritize user-specific historical data and individualized patterns when generating outputs. This enables recommendations that are tailored to individual variability rather than constrained by population-level expectations.

In some implementations, the system may iteratively refine its recommendations based on feedback from prior recommendations and observed outcomes. For example, the system may evaluate whether a prior recommendation resulted in a desired physiological response and adjust future recommendations accordingly. This feedback loop allows the system to adapt over time to individual user responses.

The disclosed embodiments are also beneficially designed to use a machine learning (ML) model to recommend continuance or modification of a usage of a GLP-1 treatment for a user. This GLP-1 treatment can be designed to treat a physiological condition of the user, such as perhaps diabetes or even perhaps obesity. To assist with that endeavor, one or more CGMs are used to monitor the user’s glucose levels. These monitored glucose levels are used to facilitate the ML model’s recommendation regarding how, when, or if to modify the GLP-1 treatment.

Although many examples refer to GLP-1 receptor agonist therapies, it should be understood that the disclosed principles may be applied to other types of therapies that influence physiological levels measured by wearable monitors. For example, the techniques may be used in connection with insulin-only therapies, combination therapies, or other pharmacological or non-pharmacological interventions. References to GLP-1 are therefore illustrative and not limiting.

The disclosed embodiments provide various improvements, benefits, and practical applications to the technical field of sensor data collection, analysis, and patient treatment. In particular, the embodiments are beneficially able to operate even when intermittent (aka “sparse” or “periodic” or “baseline”) sensor data is available. The embodiments use this intermittent data to generate recommendations on how to modify GLP-1 treatments. The embodiments can advantageously use whatever sensor data is available as an initial baseline so as to generate or modify a recommended GLP-1 treatment. This recommendation is then made available to an application. The application can then provide various insights to a user, even when sensor data is not actively being acquired. As a result, the application can be used even when an analyte sensor (e.g., the CGM) is not being worn by a user.

The disclosed embodiments beneficially enable users to better manage their lifestyles via the use of an improved application for analyte data and via the generation of recommended treatments. What this means is that the embodiments provide various mechanisms to enable all types of users, including diabetics and even pre-diabetics, to better manage their own lifestyles. As a result, significant advantages and value-adding experiences are provided to individuals who may only periodically use a sensor.

Additionally, the disclosed embodiments provide an improved user experience with respect to an analyte sensor and with respect to an application that uses data obtained from the analyte sensor. The embodiments also enable the collected sensor data and even ML generated data to be shared with other individuals and/or health professionals. Furthermore, the disclosed application can operate using the ML generated data to generate various different recommendations, scores, or even alerts. The disclosed embodiments also enable data to be analyzed to detect trends. Such trends can operate as a basis for which a recommendation is generated, such as a recommendation to visit a health care specialist.

Throughout this disclosure, there are many references to glucose levels and CGMs. A person skilled in the art, however, will appreciate how the disclosed principles can be applied to any type of analyte and to any type of analyte sensor, including both invasive sensors (i.e. a sensor that at least partially enters a patient’s body) and non-invasive sensors (i.e. a sensor that does not enter the patient’s body). Examples of analytes include, but are not limited to, glucose, ketone, lactate, alcohol, and others. Therefore, even though a majority of the examples are with respect to glucose levels and CGMs, the principles should be viewed as being more broadly applicable.

GLP-1 receptor agonists (GLP-1 RAs) are highly effective for managing type 2 diabetes (T2D), especially in individuals at risk for cardiovascular and kidney complications. However, despite their clinical benefits, GLP-1 RAs are frequently discontinued. Gastrointestinal side (GI) effects, cost, and lack of perceived benefit are some of the common reasons for stopping the GLP-1 RA therapy. Physicians may also find it challenging to prescribe GLP-1 RAs given complexity of patient profiles, side effects management, lack of time and support to closely monitor patients during titration, and limited guidelines on escalating or deescalating concurrent therapies while initiating and/or titrating GLP-1 RA.

At least some of the disclosed embodiments are beneficially designed to address both physician-specific and patient-specific challenges in GLP-1 RA therapy by leveraging data from CGMs, continuous ketone monitor (CKM), continuous lactate monitor (CLM), continuous alcohol monitor, and/or dietary information from food applications (aka “apps”). It should be understood that continuous multi-analyte monitors, may also be used (e.g., a single continuous analyte sensor for monitoring glucose, ketone, lactate, and/or alcohol).

In some implementations, analyte measurements from different sensor types may be jointly processed to generate composite features that reflect interactions between physiological systems. For example, glucose data may be evaluated in conjunction with ketone levels to distinguish between reduced food intake and improved glycemic control, or lactate levels may be analyzed alongside glucose trends to assess the impact of physical activity on metabolic response. These combined features may be provided as input to the ML model to enable differentiation between physiological states that would not be distinguishable using a single analyte alone.

For instance, at least some of the disclosed embodiments detail various features derived from continuous glucose and/or ketone monitoring data to guide and personalize GLP-1 RA treatment. These features can also beneficially be used to encourage GLP-1 RA adherence. As such, specific therapies and treatments can be implemented and facilitated by the disclosed embodiments.

In some implementations, the system may generate user-facing outputs based on relationships between multiple physiological signals and inferred user states. For example, ketone levels, lactate levels, and analyte trends may be jointly evaluated to infer conditions such as insufficient caloric intake, excessive exertion, or increased likelihood of adverse effects. Based on these inferred conditions, the system may generate corresponding guidance or alerts. This unified processing of multiple physiological indicators enables coordinated feedback rather than isolated signal-based notifications.

### Systems and devices

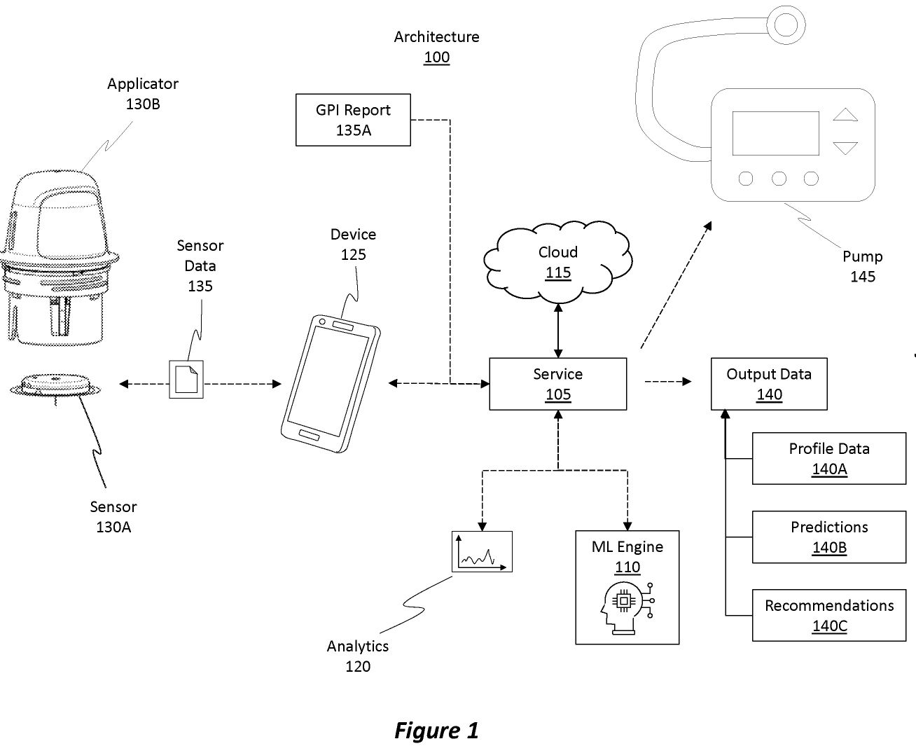

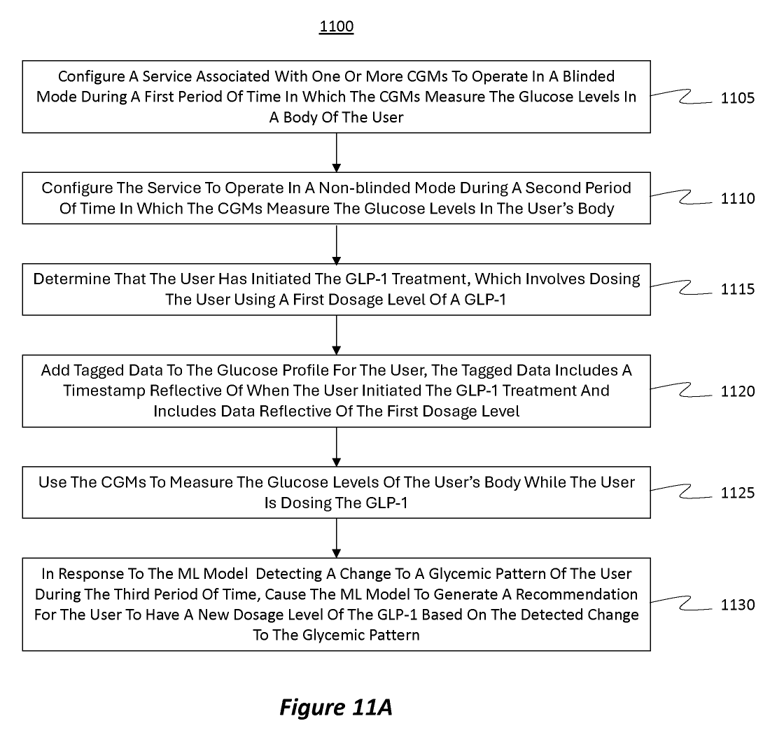

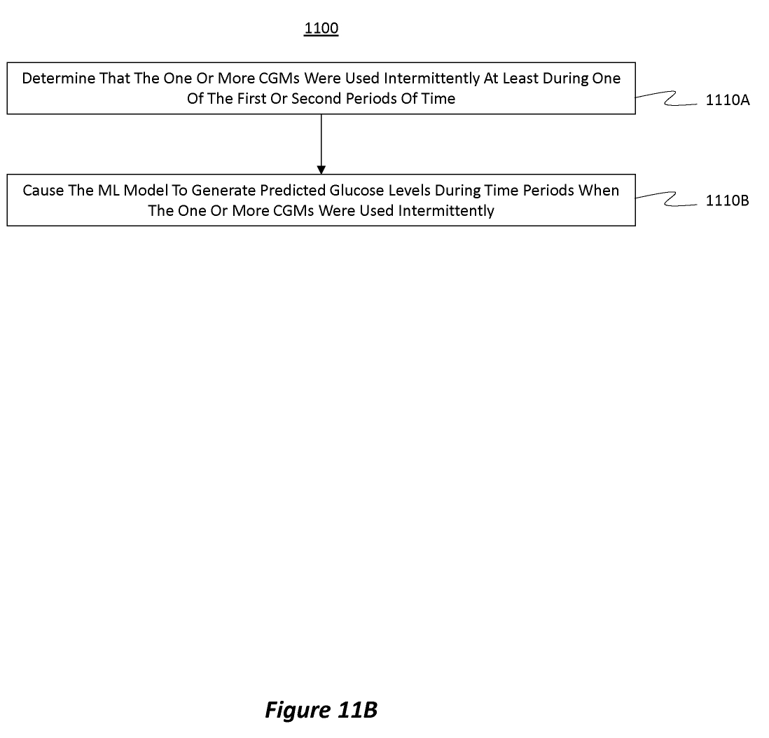

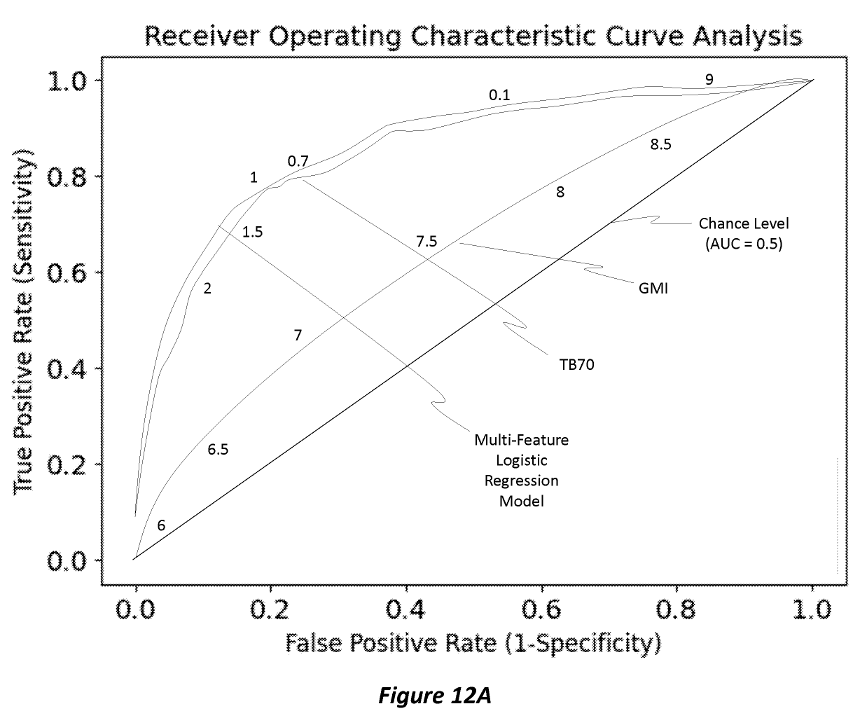

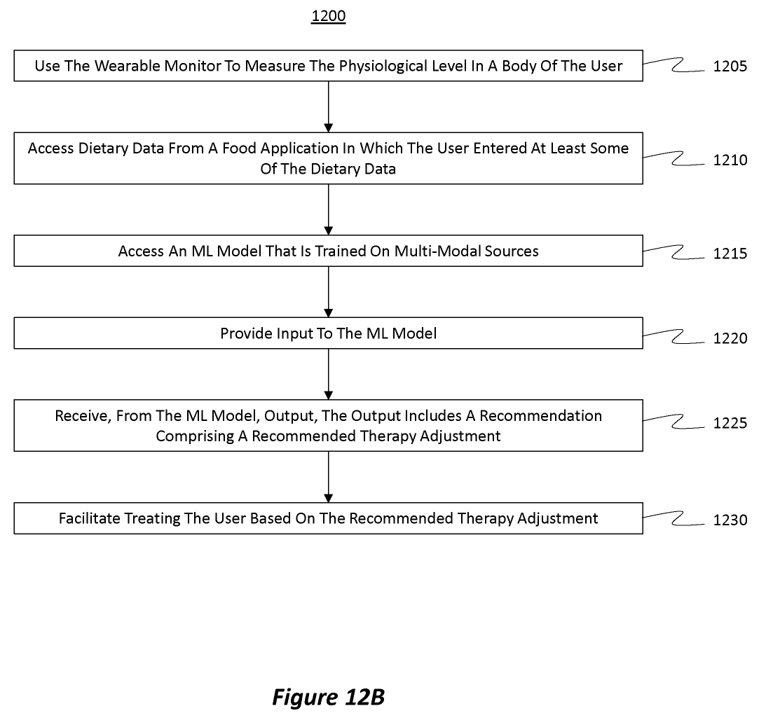

Having just described some of the high level benefits of the disclosed embodiments, attention will now be directed to FIG. 1, which illustrates an example architecture 100 that can be used to achieve these benefits. In particular, example architecture 100 may be used to implement the methods of the present disclosure, including the methods of FIGS. 11A, 11B, and 12B, .

Architecture 100 is shown as including a service 105. As used herein, the term “service” refers to an automated program (e.g. a computer program) that is tasked with performing different actions based on input. In some cases, service 105 can be a deterministic service that operates fully given a set of inputs and without a randomization factor. In other cases, service 105 can be or can include an artificial intelligence (AI) or machine learning (ML) engine, as shown by ML engine 110. With the ML engine 110, service 105 can operate even when faced with various different randomization factors.

As used herein, reference to any type of ML or AI may include any type of ML algorithm or device, convolutional neural network(s), multilayer neural network(s), recursive neural network(s), deep neural network(s), decision tree model(s) (e.g., decision trees, random forests, and gradient boosted trees) linear regression model(s), logistic regression model(s), support vector machine(s) (“SVM”), AI device(s), or any other type of intelligent computing system. Any amount of training data may be used (and perhaps later refined) to train the ML algorithm to dynamically perform the disclosed operations.

In some implementations, the ML model used to generate therapy-related recommendations may be structured to process time-series physiological data in combination with supplemental user data. For example, the model may include a first component that receives sequential analyte measurements (e.g., glucose values over time) and extracts temporal features indicative of patterns such as variability, trends, or excursions. A second component may receive non-temporal inputs, such as demographic data, medication information, or dietary data, and generate corresponding feature representations. The outputs of these components may be combined, for example through concatenation or another integration technique, and provided to a downstream component that produces an output indicative of a recommended therapy action or classification. In some scenarios, the model may be trained using labeled training data in which input data is associated with known therapy outcomes, while in other scenarios the model may be refined using user-specific data collected during operation. This description is provided to illustrate concrete example structures and data flows for ML models used in the disclosed embodiments, without requiring any particular model architecture.

ML engine 110 can initially be trained using a set of training data based on a large corpus of user information. For instance, any number of users can submit their sensor readings to a repository, which can be used to train the ML engine 110. ML engine 110 can be trained to identify trends, patterns, abnormalities, averages, or any other statistical aspect on the users’ data. Subsequently, the ML engine 110 can be further trained or fine-tuned based on a specific user’s historical data. Thus, in some scenarios, a multi-stage or multi-phase training approach can be used for training the ML engine 110.

The first step in training a machine learning model is the acquisition of a dataset. The dataset may originate from various sources, such as databases, files, APIs, or real-time sensor feeds. For example, data obtained from various sensors (e.g., by the same user or different users) or population data may be obtained. The dataset comprises multiple records, where each record includes one or more features (e.g., an independent variable). Optionally, each record includes an associated label (e.g., a dependent variable or target). This target may be used for supervised learning tasks, while unlabeled data can be used for unsupervised learning.

Once the data is collected, it undergoes a preprocessing phase to ensure that it is clean and suitable for training. Regarding data cleaning, this process involves removing or imputing missing values, eliminating duplicate entries, and correcting inconsistencies in the dataset. Regarding data normalization or scaling, the process involves transforming feature values to ensure they are on the same scale, which improves model performance in certain algorithms. Regarding categorical encoding, this process involves converting categorical variables into numerical form (e.g., using one-hot encoding) for algorithms that require numeric input.

Regarding data splitting, this process involves dividing the dataset into two or more subsets, typically training and testing sets. A portion of the data is used for model training, and the remaining portion is reserved for model evaluation to prevent overfitting.

Relevant features are selected or engineered from the dataset to improve model performance. Feature selection techniques are used to identify which attributes (e.g., features) have the most influence on the target variable. In embodiments, various feature selection techniques can be used, such as filter methods, information gain, chi-square test, fisher’s score, correlation coefficient, variance threshold, mean absolute difference, wrapper methods, forward feature selection, backwards feature elimination, exhaustive feature selection, recursive feature elimination, embedded methods, lasso regularization, random forest importance, and more.

Feature engineering can also create new features through transformations or combinations of existing ones. In embodiments, feature engineering techniques include imputation, discretization, categorical encoding, feature splitting, handling outliers, variable transformations, scaling, some of which may overlap with data preprocessing. This ensures that the model is provided with informative and non-redundant data so that training is efficient. A machine learning model is selected based on the nature of the problem and the characteristics of the data.

The training phase involves feeding the training dataset into the chosen machine learning algorithm, allowing the model to learn patterns and relationships between the input features and the output target. During this process, the algorithm iteratively adjusts its internal parameters (weights, coefficients, and the like) to minimize the error between its predictions and the actual outcomes. In supervised learning, a loss function quantifies the difference between predicted and actual values, and the model's parameters are optimized to minimize this loss. In the case of unsupervised learning, the model attempts to discover inherent patterns or groupings within the dataset.

After training, the model’s performance may be evaluated using a separate testing dataset that was not seen during training. The model's predictions are compared against the actual outcomes using various evaluation metrics. These metrics depend on the type of problem being solved. For classification tasks, metrics such as accuracy, precision, recall, F1-score, and confusion matrix are used. For regression tasks, metrics such as mean squared error (MSE), mean absolute error (MAE), and R-squared (R²) are employed. For clustering tasks, metrics such as silhouette score or Davies–Bouldin index are used to assess the quality of the clusters.

To avoid overfitting (e.g., where the model performs well on training data but poorly on unseen data), techniques such as cross-validation or regularization may be employed. To improve model performance, hyperparameters (e.g., predefined settings that control the learning process, such as learning rate or number of estimators) can be fine-tuned. This process can involve manual tuning, grid search, or random search methods. Cross-validation may be used in this step to ensure that the tuned model generalizes well to new data.

Once the model achieves satisfactory performance, it is ready for deployment. Deployment refers to integrating the trained model into an application or system where it can make predictions on new, unseen data. The data input to the trained model generally mirrors the data used to train the model. In some embodiments, the model may be exposed as an API, embedded into a software application, or used in batch processing pipelines. The deployed model may be monitored over time to ensure it continues to perform as expected and does not degrade due to changes in input data or system environments.

As new data becomes available, the model may be retrained (e.g., personalized based on an individual’s data or refined with new population data) periodically to maintain or improve its performance. Retraining can be initiated based on temporal intervals, performance thresholds, or changes in the data distribution. In some cases, models are continuously updated in real-time (online learning) to adapt to new data.

In some implementations, personalization of the model may occur incrementally over time as additional user-specific data is collected. For example, early recommendations may rely more heavily on population-level data, while later recommendations may place greater emphasis on the user’s historical patterns and responses to prior therapies. The relative weighting between population-derived knowledge and individual-specific data may shift dynamically as the amount and quality of user-specific data increases. This progressive personalization can enable the system to adapt to individual variability without requiring explicit retraining events visible to the user.

By way of a more detailed example, in some implementations the ML model may be trained using a dataset that includes time-series physiological data collected from wearable monitors across a plurality of users. For instance, glucose measurements may be obtained at periodic intervals (e.g., every few minutes) over extended durations (e.g., multiple days or weeks), thereby forming glucose profiles that capture temporal patterns. These profiles may be combined with associated contextual data, such as insulin dosing records, GLP-1 RA usage information, dietary inputs, and clinical outcomes. In some scenarios, the training data may be labeled to indicate outcomes of interest, such as whether a user experienced hypoglycemia, whether a therapy adjustment was made by a clinician, or whether a user exhibited a favorable response to a therapy. Labeling may be performed based on retrospective clinical records, rule-based criteria (e.g., detection of events within the physiological data), or a combination thereof.

In some implementations, the model architecture may include a neural network configured to process both time-series and non-time-series inputs. For example, a first portion of the model may include one or more layers adapted for sequential data (e.g., recurrent layers or temporal convolution layers) to process the physiological measurements, while a second portion of the model may include one or more fully connected layers to process static or contextual inputs (e.g., demographic or medication data). The outputs of these portions may be combined and provided to one or more additional layers that produce an output indicative of a recommendation, classification, or predicted outcome. In some scenarios, the model may instead use alternative architectures, such as tree-based models or regression models, depending on the nature of the input data and the prediction task.

Training of the model may be performed using an objective function that reflects the intended prediction task, such as a loss function measuring error between predicted and observed outcomes. The training process may include iteratively updating model parameters over multiple passes through the training dataset, and may optionally include validation steps using separate data to assess performance. In some implementations, training may be performed using population-level data and later refined using user-specific data collected during operation. This example is provided to illustrate one manner in which the ML model may be concretely implemented and trained using the types of data described herein, while other variations may also be used.

In some implementations, service 105 is a cloud service operating in a cloud environment, such as cloud 115. In some implementations, service 105 is a local service operating on a local device (e.g., sensor 130A, device 125, and/or any other device). In some implementations, service 105 is a hybrid service that includes a cloud component operating in the cloud 115 and a local component operating on a local client device. These two components can communicate with one another.

Service 105 is tasked with various operations that include collecting sensor data, analyzing that sensor data, and determining the impact of that sensor data with respect to a user associated with the sensor data. To do so, service 105 can include an analytics 120 component that is capable of performing data analysis on the collected sensor data. In some examples, analytics 120 component and ML engine 110 can be the same component.

As shown in FIG. 1, service 105 can communicate with a device 125. Device 125 can be any type of personal device, including any type of wearable device or mobile device. Examples of device 125 include, but certainly are not limited to, any type of device reader, smart phone, tablet, laptop, desktop, wearable device, and so on. Device 125 is shown as communicating with a sensor 130A and is further shown as receiving sensor data 135 from the sensor 130A. Sensor 130A is the component that collects the sensor data 135. In some cases, device 125 and sensor 130A can be implemented on the same device. In some implementations, sensor 130A is initially included as a part of an applicator 130B that is used to adhere or connect the sensor 130A to a user’s body.

Sensor 130A can be any type of sensor. One particular example of sensor 130A includes a CGM. A CGM operates by inserting a small sensing unit under a person’s skin. This sensing unit then measures that person’s interstitial glucose levels. Typically, this sensing unit acquires new data at a periodic rate, such as once every selected number of minutes (e.g. 1 minute, 2 minutes, 5 minutes, 10 minutes, 30 minutes), although the data could also be collected continuously. That data is represented as sensor data 135 in FIG. 1.

Device 125 communicates with sensor 130A using any type of near-field wireless communication technology, such as BLUETOOTH. As a result, sensor data 135 is transmitted from sensor 130A to device 125 over that communication protocol. Service 105 can communicate with device 125 via any type of wireless communication protocol as well. In some implementations, the communication protocol is a BLUETOOTH protocol. In some implementations, the protocol is a wireless fidelity (Wi-Fi), near field communication (NFC), and/or Internet Protocol (IP). Often, device 125 transmits the sensor data 135 to the cloud 115, where that sensor data 135 is then stored in a repository that is accessible to service 105.

In some implementations, the sensor data 135 is encrypted or otherwise integrity protected to ensure tampering does not occur. Also, in some implementations, any personally identifiable information (PII) is stripped from sensor data 135 prior to it being stored in the cloud 115.

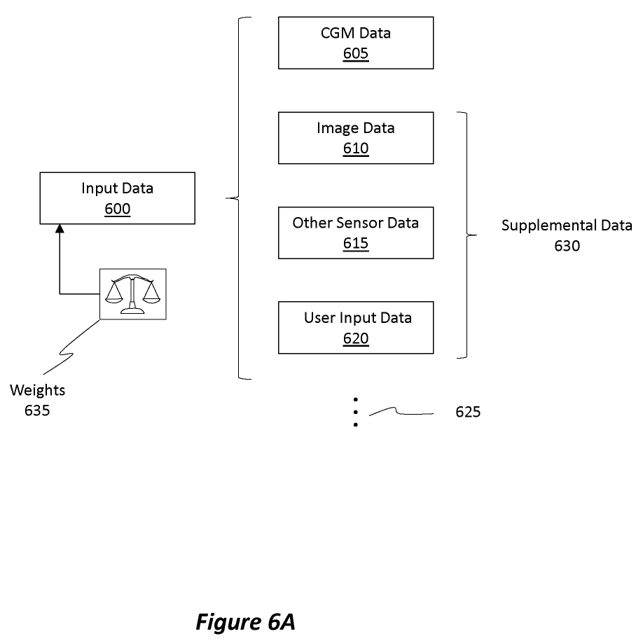

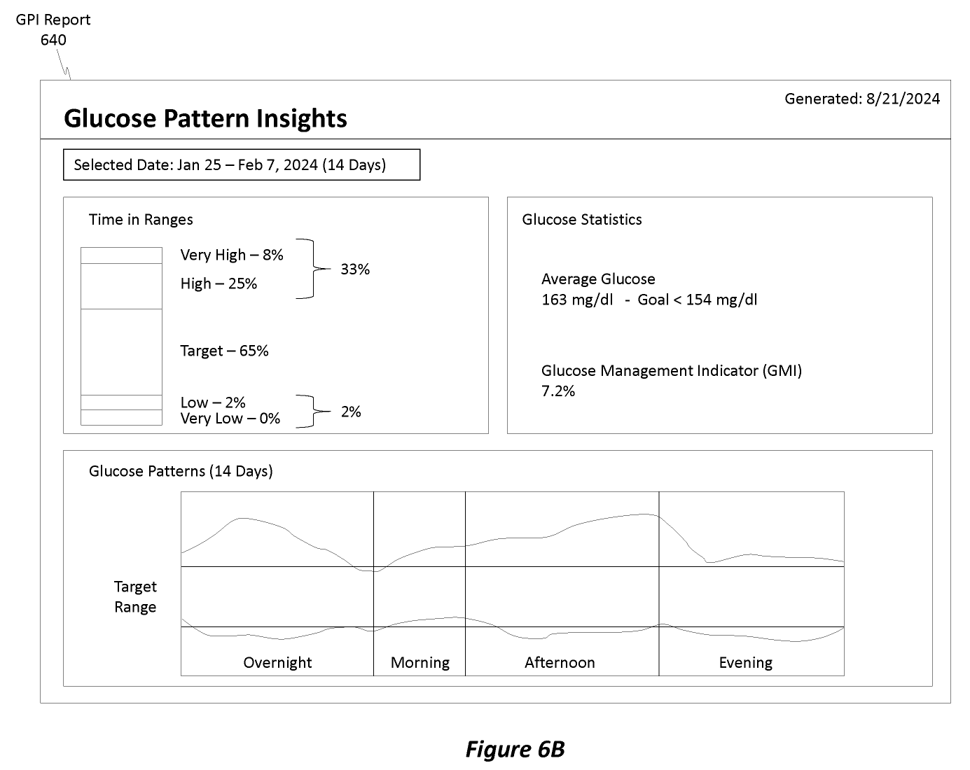

In some scenarios, service 105 can also acquire a glucose pattern insights (GPI) report 135A and/or data associated with GPI report 135A. The GPI report 135A includes information that can help interpret a user’s sensor data, such as the CGM data. By way of example and not limitation, the report can include statistics on overall glucose levels for specific categories of users (e.g., males at a given age range and weight range, as one example), medication guidelines, and even lifestyle considerations. Different summaries or patterns on lifestyle or physiological conditions can also be described in the report. An example of a GPI report is provided in FIG. 6B and will be discussed in more detail later.

Service 105 uses its analytics 120 component and/or the ML engine 110 to analyze the sensor data 135 as well as potentially the GPI report 135A. Service 105 generates output data 140 as a result of performing that analysis. In the scenario where sensor 130A is a CGM and where the sensor data 135 reflects glucose levels for a user, the output data 140 can reflect glycemic insights such as a glycemic impact for the user. Glycemic impact generally refers to a patient’s bodily state with respect to blood sugar levels.

In some scenarios, the output data 140 can include profile data 140A for the user, predictions 140B for the user, and even ML generated recommendations 140C for the user. Additionally, in some scenarios, service 105 can modify the operation of a user’s insulin pump 145 to adjust the amount of insulin the user may be getting at any given time. The modification can also occur by titrating basal and/or prandial insula doses to be administered using an injection pen.

In some implementations, the recommendation may be provided in different forms depending on the context in which it is used. For example, the recommendation may include a direct dosage adjustment suggestion, a ranked set of alternative therapy options, a confidence score associated with a suggested adjustment, or a notification indicating that further monitoring is recommended prior to making a change. In other cases, the recommendation may be presented as a trend-based insight (e.g., indicating that current therapy is trending toward a particular outcome) rather than as a discrete adjustment. These different forms of output can be selectively generated based on available data, user preferences, or clinical workflows.

Advantageously, service 105 is generally tasked with using a machine learning (ML) model (e.g., ML engine 110) to recommend continuance or modification of a usage of a glucagon-like peptide-1 (GLP-1) treatment of a user, where the GLP-1 treatment is designed to treat a physiological condition of the user and where one or more continuous glucose monitors (CGMs) (e.g., sensor 130A) monitor glucose levels of the user. Beneficially, the monitored glucose levels are used to facilitate the ML model’s recommendation (e.g., recommendations 140C) regarding continuance or modification of the GLP-1 treatment.

In contrast to approaches that derive therapy guidance based on inferred gastric emptying characteristics or proxy physiological rates, the disclosed embodiments do not require estimation of gastric emptying or similar intermediary physiological processes. Instead, the disclosed embodiments operate directly on observed and derived features from analyte measurements, dietary inputs, and user-specific data to generate recommendations. This allows the system to identify relationships between inputs and outcomes without relying on intermediate physiological modeling assumptions.

To achieve the above, service 105 can optionally be configured to operate in a “blinded mode” during a first period of time in which the one or more CGMs measure the glucose levels in the user’s body prior to initiating GLP-1 therapy. Alternatively, service 105 can be configured to not operate in the blinded mode and instead simply operate in the non-blinded mode. Service 105, when operating in the blinded mode, prevents display (i.e. to a user) of measurements obtained by the CGMs during the first period of time. The measured glucose levels obtained during the first period of time are included in a glucose profile for the user, such as within profile data 140A of FIG. 1.

One reason why it may be beneficial to operate in the blinded mode is because users will often modify their normal behavior if they are made aware of their actual glucose levels. For instance, if a user sees a sudden spike in their levels, that user will often later modify his/her behavior to avoid a similar kind of spike. This behavior modification can skew the user’s data, resulting in service 105 not being able to acquire an accurate baseline report for the user. Thus, the blinded mode can help service 105 obtain an accurate understanding of the user’s normal behavioral patterns. In some examples, the blinded mode may be bypassed (e.g., when the user is already a CGM user or when the user is already a GLP-1 user).

It should be understood that “blinded mode” does not necessarily require complete suppression of all information presented to the user. In some scenarios, limited or abstracted feedback may still be provided, such as generalized status indicators, delayed summaries, or non-numeric cues that do not reveal specific analyte values. In this manner, the system can reduce the likelihood of user-driven behavioral modification while still maintaining user engagement with the application. The degree of information suppression or abstraction may vary across implementations.

In some implementations, data collected during the blinded period may be treated as a baseline dataset with reduced user-induced behavioral bias and may be weighted differently from data collected during non-blinded periods. For example, the system may assign higher confidence or influence to blinded-period data when establishing initial model parameters or baseline patterns, thereby improving robustness of subsequent comparisons and recommendations.

Subsequent to the first period of time, service 105 is configured to operate in a non-blinded mode during a second period of time in which the one or more CGMs measure the glucose levels in the user’s body prior to initiating GLP-1 therapy. When operating in the non-blinded mode, service 105 enables display of measurements obtained by the CGMs during the second period of time. The measured glucose levels obtained during the second period of time are included in the glucose profile for the user. Because service 105 has already obtained accurate data regarding the user’s behavior (due to operating initially in the blinded mode or prolonged CGM use) and because the user has already been subjected to wearing the sensor for a period of time, data obtained while service 105 operates in the non-blinded mode is less likely to be skewed.

In some scenarios, however, service 105 can operate in the blinded mode throughout both the first and second time periods, such that there is essentially no distinction between the two time periods (i.e. there is essentially only a single time period). Similarly, in some scenarios, service 105 can operate in the non-blinded mode throughout both the first and second time periods. For example, if the user is already a CGM user, the first and second time periods can correspond to the period of time prior to starting GLP-1 treatment. Accordingly, use of the blinded and non-blinded modes are simply one option for configuring service 105, and those different modes are not a requirement.

In some implementations, the delineation between time periods may be reduced, expanded, reordered, or omitted entirely. For example, a system may operate using a single continuous monitoring period without distinguishing between baseline and post-baseline phases. In other scenarios, additional periods may be introduced, or the duration of any period may vary dynamically based on data availability or user behavior. Accordingly, the described time periods represent one example organizational structure for data collection and analysis.

Subsequent to the second period of time, service 105 determines that the user has initiated the GLP-1 treatment. This determination can be made in a variety of ways. As one example, service 105 can examine the recorded sensor measurement data and infer that, due to a threshold level of change in the user’s glucose patterns relative to the previous baseline pattern, the user likely started a new treatment (i.e., the GLP-1 treatment). As another example, the user can provide information to the service 105 indicating the start of the new treatment. As another example, the user’s electronic medical records can optionally be accessed, and the medical records may reveal the user’s prescription for GLP-1. As another example, the determination can be based on examining data that has been tagged to indicate a new treatment has occurred. Additionally, or alternatively, the determination can occur by service 105 examining changes in patterns or trends to the user’s CGM data or other data.

The GLP-1 treatment involves dosing the user using a first dosage level of a GLP-1. This first dosage level may be a level selected for the specific user or it may be a default selection (e.g., by the users HCP). In some scenarios, the initial dose may be determined by the system using a population model, the first and second period data, prior use of GLP-1s, and/or the user’s goals (e.g., lose weight, lower A1C/GMI levels).

Optionally, in some scenarios, the CGM can be used concurrently with GLP-1s from the start. For instance, instead of having the different time periods prior to the start of the GLP-1, some embodiments monitor the user’s glucose levels immediately or shortly after the user starts a GLP-1 treatment.

Service 105 can also add tagged data to the glucose profile for the user. The tagged data includes a timestamp reflective of when the user initiated the GLP-1 treatment. The tagged data can also include data reflective of the first dosage level as well as the specific type of GLP-1 that is being used by the user. The embodiments can also tag each subsequent dose.

During a third period of time subsequent to the second period of time, service 105 uses the CGMs to measure the glucose levels of the user’s body while the user is taking the GLP-1. The measured glucose levels obtained during the third period of time are included in the glucose profile for the user. Optionally, service 105 can detect a specific change in the user’s glycemic pattern as compared to the user’s previous pattern. Service 105 can determine the percentage difference that exists between the two patterns.

In response to the ML model detecting a change to a glycemic pattern of the user during the third period of time, service 105 causes the ML model to generate a recommendation for a particular dosage level of the GLP-1. This recommendation is based on the detected change to the glycemic pattern. In some scenarios, the recommendation may be that the user continue the current dosage for at least a predetermined time period, thus the “particular” dosage level is the same as the previous dosage level. In some scenarios, the recommendation can further include a titration (up or down) after the predetermined time period elapses. In yet other scenarios, the recommendation may include a different and new dosage level than what was previously used by the user.

In some implementations, the generation of a recommendation may be triggered by conditions other than or in addition to a detected change in glycemic pattern. For example, recommendations may be triggered based on elapsed time, user input, detection of specific events (e.g., missed meals, medication changes), threshold-based conditions, predicted future states, or combinations thereof. In some cases, the recommendation may be generated continuously or periodically independent of any specific detected change.

In some implementations, recommendations are not generated based on fixed threshold comparisons or predefined trigger conditions. Instead, the ML model may evaluate combinations of features and learned relationships to produce outputs that are not reducible to simple threshold exceedances. For example, the model may generate different recommendations for similar measured values depending on contextual factors such as prior trends, user behavior, or multi-modal inputs.

Optionally, service 105 can include an option to indicate whether the user has type 1 diabetes, has type 2 diabetes, is on an oral medication, or is on insulin (e.g., basal or prandial insulin). Service 105 can make these indications based on a review of the user’s tagged data, sensor data, medication listing, user data, or any other data. In some scenarios, the recommendation for the new dosage of GLP-1 can be based on this particular user indication.

Also, the ML model of service 105 can be trained to identify one or more of the following detected conditions: a first condition in which an A1C response of the user lowers (e.g., in response to dosing the GLP-1) but a weight of the user does not lower, or perhaps use of a glucose management indicator (GMI), which is a metric calculated as an “approximate A1C level based on average CGM glucose levels” in a GPI report; a second condition in which the weight of the user does lower (e.g., in response to dosing the GLP-1) but the A1C response does not lower; or the user is one who does not respond to GLP-1 type medications (e.g., little to no change in weight, GMI, and/or A1C). Optionally, service 105 can perform these identifications without having to rely on machine learning. Service 105 can also determine or identify other insights, which can be user goals or other recommendations, conditions, or criteria.

In some implementations, the ML model uses the glucose levels measured by the one or more CGMs to predict whether the user is one who will respond earlier than a specific week (e.g., perhaps a 4 week) titration period. The prediction of the ML model is based on a detected lack of a glycemic response from the user while the user is being treated with the GLP-1. For instance, the prediction may indicate that the user will likely respond within 1 week, 2 weeks, or perhaps 3 weeks. In some scenarios, the prediction may indicate that the user will likely take longer to respond than normal, such as perhaps 5 weeks, 6, weeks, 7 weeks, 8 weeks, or longer than 8 weeks.

Service 105 can also be configured to monitor a gastrointestinal tolerance for the user while the user is being treated with the GLP-1. For instance, service 105 can receive user feedback indicating the user’s tolerance. If the user is not tolerating the current dosage well, service 105 can generate a modified recommendation regarding the dosage. Often, the modification is a reduction in the dosage amount.

In some implementations, recommendations may be generated independently of reported symptoms or subjective inputs. For example, recommendations may be based solely on measured or derived physiological features without requiring the user to report symptoms such as gastrointestinal discomfort. This allows the system to operate even in the absence of subjective symptom data.

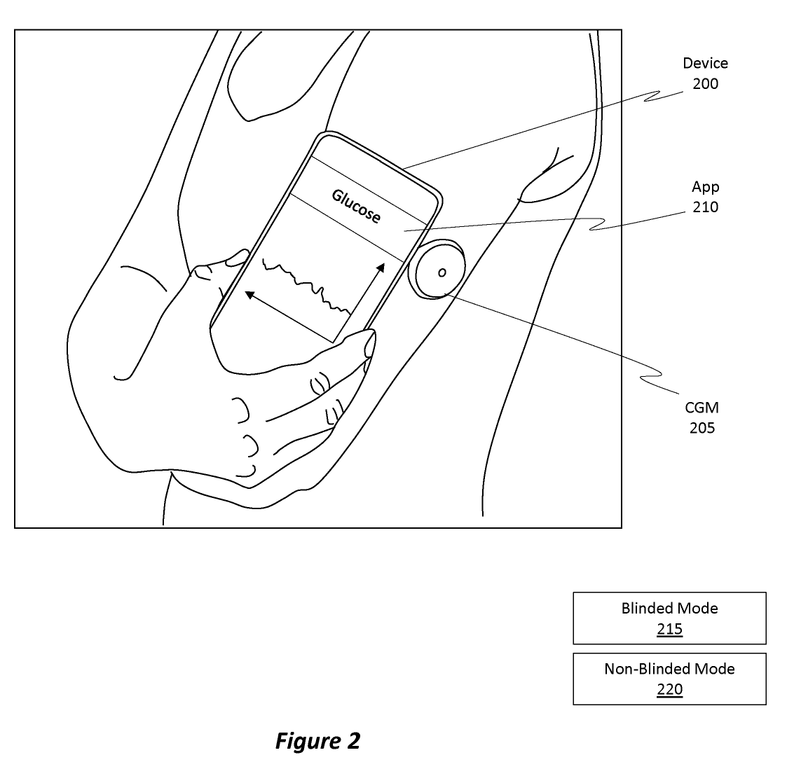

FIG. 2 shows a device 200, which is representative of device 125 from FIG. 1. Device 200 is in communication with a CGM 205, which is representative of sensor 130A. In FIG. 2, CGM 205 is currently affixed to the user’s arm and is tracking the user’s glucose levels. Device 200 is hosting an application (or simply “app”) 210. App 210 provides a visualization of the tracked data (e.g., a graph or trace of glucose levels over a period of time).

In some scenarios, the app 210 (e.g., which can be implemented by service 105 of FIG. 1) can operate either in a blinded mode 215 or a non-blinded mode 220. Thus, the app 210 and the service 105 can operate in a blinded or non-blinded mode.

While operating in the blinded mode 215, the app 210 can purposefully hide the obtained sensor data from the user for a limited period of time (e.g., a select number of days or weeks). As mentioned earlier, hiding the data from the user can be advantageous in some scenarios because when the data is readily available to a user, some users will modify their behaviors based on the data, potentially resulting in obscured, skewed, or inaccurate data. If a user is not apprised of the data, then the likelihood that the user will modify his/her behavior can be reduced. While operating in the non-blinded mode 220, the app 210 can provide the sensor data to the user. In some examples, blinded mode 215 may be bypassed (e.g., when the user is already a CGM user or when the user is already a GLP-1 user). Further details on these aspects will be provided later.

### Continuous glucose monitor data

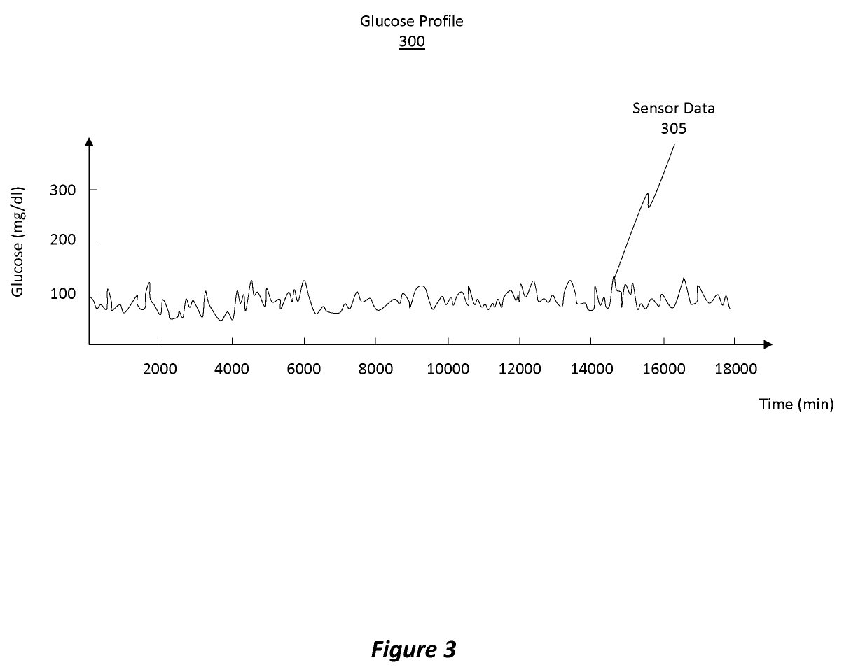

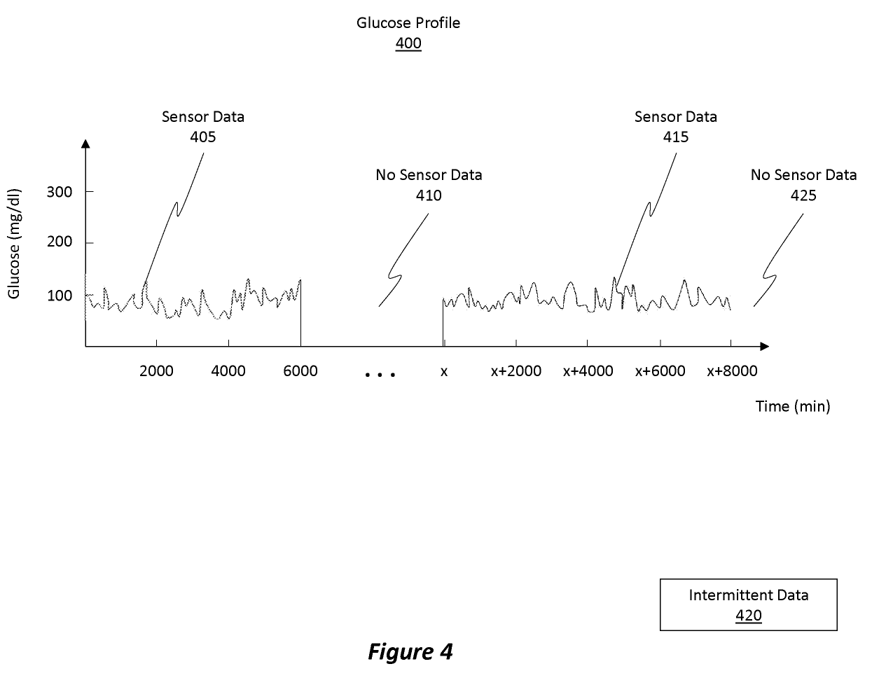

FIG. 3 shows a glucose profile 300 (also referred to herein as glucose chart 300), which can be displayed by the app 210 of FIG. 2. Glucose chart 300 is displaying the sensor data 305, which is reflective of the user’s glucose levels over a period of time. For instance, the horizontal axis reflects the glucose levels over a minutes-based time period. It should be noted that, although glucose profile 300 may not be truly continuous in the sense that the sensor data 305 is acquired at a periodic rate (such as once every selected number of minutes), for the purpose of the present disclosure it is still considered continuous, especially when compared to the intermittent scenario set out with respect to FIG. 4. In general, a glucose profile 300 may be considered continuous as long as the sensor data 305 is acquired at the periodic rate without stopping obtaining data.

In some scenarios, the user might not currently be wearing an active CGM sensor. As a result, device 125 of FIG. 1 is not able to obtain updated sensor data and does not display glucose information.

FIG. 4 shows a glucose profile 400 that includes first sensor data 405 obtained during a first time period a first CGM sensor. At time 6,000, the user stopped wearing the first CGM sensor, or the CGM stopped obtaining data for some reason (e.g., the sensor expired, fell off, was damaged). Thus, there is a period of time 410 during which no sensor data is obtained. During this time period, the historical usefulness of app 210 from FIG. 2 is significantly reduced, or ceases all together, because the functions of app 210 rely on up-to-date sensor data.

Later, second sensor data 415 is again obtained (e.g., using the first CGM sensor or a second CGM sensor, different than the first sensor). For instance, the user may have obtained a new sensor that now works, or the user reaffixed the sensor to his/her arm. FIG. 4 thus shows a scenario in which intermittent data 420 (e.g., sensor data 405 and 415) (aka sparse data or missing periodic data) is obtained. Later, there is another period of time 425 in which no sensor data is obtained. The disclosed embodiments are able to operate even when using intermittent data. In accordance with at least some of the disclosed principles, the embodiments can generate predicted data for the time periods during which no sensor data was obtained. This predicted data can then fill in for those missing time periods.

In some implementations, the system may be configured such that recommendations are generated without requiring continuous analyte monitoring. For example, the ML model may operate on a combination of historical measured data and predicted data representing unmeasured intervals, thereby enabling analysis across discontinuous datasets. This allows the system to function in scenarios where continuous sensor usage is not available or not maintained.

In some implementations, the system is configured to generate recommendations using data collected during non-contiguous monitoring periods. For example, sensor data collected during separate wear intervals may be combined and analyzed to generate recommendations, without requiring continuous monitoring. This allows the system to operate in scenarios where sensors are used intermittently rather than continuously.

In some optional implementations, intermittent sensor usage may be intentional rather than incidental. For example, a user may periodically wear a sensor for defined intervals separated by periods without active sensing. During these intervals without sensor data, the system may rely more heavily on previously observed patterns, supplemental data sources, or predicted values to maintain continuity in the user’s profile. When new sensor data becomes available, the system may reconcile the newly acquired data with the previously predicted or inferred data to refine subsequent analyses. This approach allows flexibility in sensor usage while still enabling meaningful insights and recommendations.

Predicted data may be stored and represented in a manner that distinguishes it from directly measured sensor data. For example, predicted values may be tagged, flagged, or otherwise encoded with metadata indicating their inferred nature. This distinction allows downstream processing components to selectively include or exclude predicted data when generating recommendations or visualizations. In some scenarios, predicted data may be updated or replaced as new sensor measurements become available, thereby maintaining consistency within the user’s data profile.

In some implementations, predicted data may be used independently of measured data to generate recommendations, even when measured data is available. Conversely, predicted data may be omitted entirely in scenarios where only measured data is used. In this way, prediction-based processing may be optional and selectively applied.

In some implementations, handling of missing or intermittent data may be performed using one or more defined data processing techniques prior to or during application of the ML model. For example, when one or more expected sensor measurements are absent, the system may generate substitute values based on interpolation between available measurements, extrapolation from prior trends, or estimation using a model trained on historical data from the same user and/or from a population of users. In some scenarios, the system may also generate one or more features that explicitly indicate the presence, duration, or frequency of missing data, and these features may be provided as input to the ML model. In this manner, the ML model may be trained to interpret both observed measurements and inferred or incomplete data conditions. In some implementations, missing data handling may be performed as a preprocessing step prior to model input, while in other implementations the model itself may be configured to accept sequences with missing entries and internally account for such gaps when generating predictions or recommendations. This enables the system to continue producing outputs even when sensor data is intermittently unavailable.

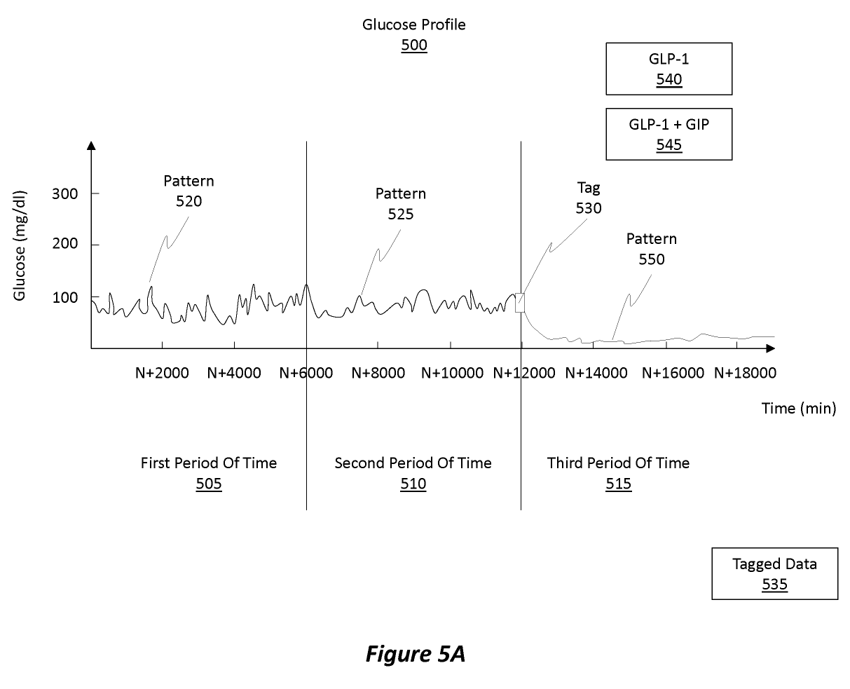

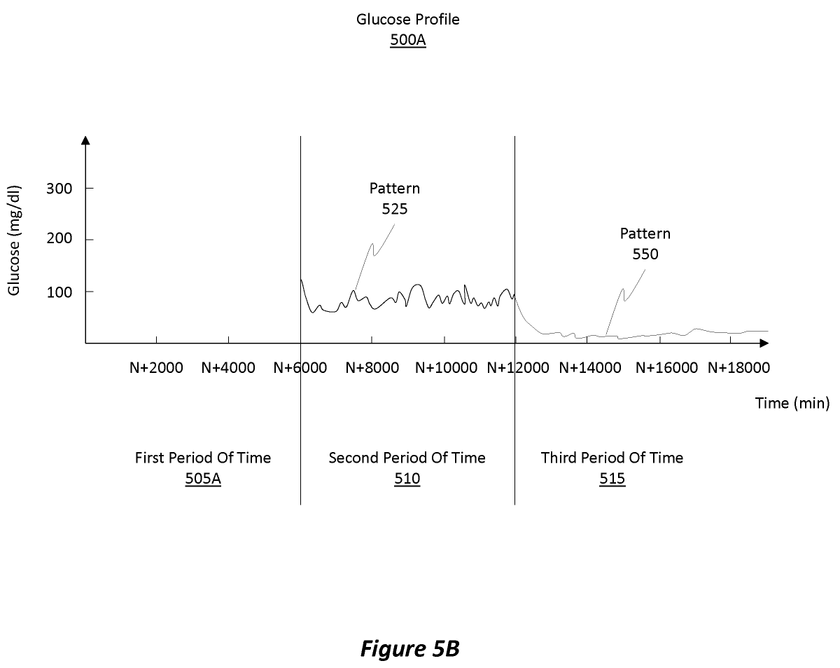

FIG. 5A shows an example glucose profile 500 that shows sensor data during three different periods of time, as reflected by first period of time 505, second period of time 510, and third period of time 515. These different time periods are reflective of the time periods mentioned earlier with respect to service 105. In some example scenarios, the first period of time 505 is a minimum of 14 days. The second period of time 510 can also be a minimum of 14 days while the third period of time 515 may be any time period. Thus, in some scenarios, the one or more CGMs are worn by the user for at least 28 day prior to the user being treated with the GLP-1 for a first time. Optionally, after either one or both of the first and second time periods finish, the service triggers a questionnaire for the user to complete. This questionnaire can be used by the user to provide feedback to service 105, where the feedback can indicate activities performed by the user, tagging information to tag specific peaks or valleys in the data, medication used, and so on. Optionally, the questionnaire can include questions about goals, current weight, goal weight, weight loss progress, etc. Also, the user can log goal weight and current weight via an app. Also, the entered current weight can be associated with a glucose trace.

During the first period of time 505, the user’s glucose levels are monitored, and a pattern 520 can be detected based on those glucose levels. Optionally, during this first period of time 505, the user’s app (e.g., app 210 from FIG. 2) can operate in the blinded mode 215 so that the user is not apprised of the data or the pattern 520. If the user is already a CGM user, first period of time 505 may operate in non-blinded mode 220. The data collected during this first period of time 505 can be viewed as being the user’s default or baseline data.

During the second period of time 510, the user’s glucose levels are continued to be monitored, and a pattern 525 can be detected based on those glucose levels. Optionally, during this second period of time 510, the user’s app can operated in the non-blinded mode 220 so that the user is apprised of the data or the pattern 525. The data obtained during this second period of time 510 can be supplemental data added to the user’s baseline data.

At the commencement of the third period of time 515, the user may tag 530 the initial set of data, resulting in tagged data 535 being generated. This tag 530 can be used to indicate that the user is starting a new treatment, such as the use of GLP-1 540 or a combination of GLP-1 + gastric inhibitory polypeptide (GIP) 545.

Notice, during the third period of time 515, the sensor data reveals a different pattern 550 as compared to patterns 520 and 525. Thus, the use of the treatment pharmaceutical is having a recognizable impact on the pattern or trend of the user’s glucose levels. Accordingly, in some scenarios, the embodiments obtain approximately 2 weeks of blinded data and approximately 2 weeks of non-blinded data. The user then starts GLP-1 for two or more weeks, and the user can see the resulting data. In some scenarios, the user may be using a GLP-1 before the user starts to use a CGM. Thus, the disclosed principles can be employed in a scenario where the user is already using a GLP-1 but has not yet started to use a CGM. In some scenarios, the user may be currently using a CGM. In some scenarios, the user may have previously been using a CGM but stopped for a period of time. The disclosed embodiments cover those scenarios as well. FIG. 5B shows an example glucose profile 500A that reflects a scenario where the user is already on GLP-1 but is not initially using a CGM during the first period of time 505A.

### Other input data

In addition to the CGM’s sensor data, other data can be used to help the ML engine 110 generate or modify treatment protocols or recommended protocols. For instance, FIG. 6A shows supplemental data 630 forming part of input data 600 that can optionally be provided to service 105 from FIG. 1 to assist in generating the ML generated recommendation. Input data 600 includes CGM data 605, image data 610, other sensor data 615, and user input data 620. The ellipses 625 demonstrates how other data can be provided to service 105 as well, such as manual input or connecting data communications with other devices or applications. The image data 610, other sensor data 615, and the user input data 620 are examples of supplemental data 630 that can be provided to service 105.

In some implementations, the system may operate using only a subset of the available input data types. For example, recommendations may be generated using only physiological sensor data, only dietary data, or a combination of two or more data sources without requiring all described inputs to be present. In certain scenarios, missing data types may be omitted without substitution, while in other scenarios, missing data may be approximated, inferred, or replaced using previously collected data or population-level data. This flexibility allows the system to operate under varying data availability conditions.

In some implementations, the system may generate recommendations based on joint interactions between multiple input modalities rather than treating each input independently. For example, the relationship between dietary intake and analyte response may be evaluated in combination with medication data and user-specific behavioral patterns. This multi-modal interaction enables the system to capture dependencies that are not observable from analyte data alone.

FIG. 6A also shows different weights 635. Different weights 635 can be applied to the different types of input data 600. For instance, the actual sensor or analyte data (e.g., CGM data 605) may be provided a highest weight while the supplemental data 630 may be provided lower weights. In some implementations, different weights can be used for different time periods and/or different conditions (e.g., bodily conditions of a patient or perhaps even environmental conditions). Weights for supplemental data can optionally change depending on the availability of CGM data. Similarly, weights for current CGM data may be highest whereas weights for baseline data may change over time until eventually that baseline data is determined to be stale. These weights can be used to later generate a score for the user, where that score can then be included in the user’s health model or profile. This score provides the user with a quick snapshot or overview of the user’s health. These weights can also impact the recommendation generated by service 105.

In some implementations, the relative importance of different data sources may be dynamically adjusted over time or based on context. For example, certain data sources may be temporarily excluded, emphasized, or replaced depending on their availability, reliability, or relevance to a particular analysis. In other scenarios, equal weighting or no explicit weighting may be applied.

FIG. 6B shows one example of a GPI report 640, which is representative of the GPI report 135A from FIG. 1 and which can be included as a part of the input data 600 from FIG. 6A. This report can document and detail various health metrics, averages, and statistical trends for users, such as A1C values or GMI values (approximate A1C level based on average CGM glucose levels). The GPI report 640 can also outline various metrics regarding the use of GLP-1s. In some examples, the data contained within GPI report 640 may be used without the report itself (e.g., a calculated GMI value may be determined or obtained outside of the GPI report). In some scenarios, the GMI value is calculated but the report itself need not be generated or used. The GMI value is, in some scenarios, the more relevant metric as compared to the report.

As mentioned, in some scenarios, the supplemental data can be used to generate a health model or profile, such as profile data 140A from FIG. 1. Optionally, different weights may be applied to the sensor data (e.g., the intermittent, sparse, or periodic sensor data) and the supplemental data. For instance, supposing the periodic sensor data includes glucose level data, the glucose level data may be provided a highest weight as compared to weights provided to the supplemental data.

Regarding the image data 610 in FIG. 6A, service 105 is able to receive images of a user’s food choices and other activities. Service 105 includes logic to analyze image content to determine what type of food a user is consuming and/or what type of activity a user is involved in (based on image data or any other data such as data from a fitness tracker). This logic enables service 105 to predict or estimate the nutritional value (e.g., macronutrient information) and ingredient list for the food and/or estimate the health aspects of a given activity. This data can also be used to generate the ML generated recommendation. The recommendation can, optionally, include recommended food choices or food changes.

In some implementations, recommendations relating to food choices or dietary changes may be generated using a model trained on associations between dietary inputs and corresponding physiological responses. For example, training data may include paired records of food intake (e.g., meal composition, macronutrient content, portion size, or food type) and resulting analyte responses (e.g., postprandial glucose profiles) collected from one or more users over time. In some scenarios, these data may be labeled or annotated to indicate desirable or undesirable outcomes, such as stable glucose levels, elevated excursions, or improved time in range. The model may be trained to identify patterns linking particular food characteristics or eating behaviors to these outcomes. During operation, when a user logs or captures information about a food item, the system may extract features (e.g., estimated macronutrient content or food category) and provide those features as input to the model. The model may then generate a recommendation by predicting an expected physiological response and selecting food options or modifications associated with more favorable predicted outcomes. In some implementations, these recommendations may be further refined using user-specific historical responses to similar foods.

In some implementations, dietary features derived from image data or logged inputs may directly influence the recommendation generation process by modifying predicted physiological responses. For example, the ML model may simulate or estimate the effect of alternative dietary actions (e.g., substituting a food item, modifying portion size, or altering intake order) and compare the predicted outcomes. The recommendation may then be selected based on the relative differences between these predicted outcomes.

Optionally, a timestamp can be provided with the image. If CGM data is available for that corresponding time stamp, service 105 is able to use the image data to provide context as to why the CGM data is what it is at a given instant in time. As an example, suppose a user’s glucose levels spiked during a particular time period. The embodiments are able to access GPS or other location data to determine where the user was when the spike occurred. As an example, the GPS data may reveal that the user was physically located in an ice cream shop. Based on this location data, the embodiments can infer that the user was likely eating ice cream, hence the spike in blood sugar levels. The above example can be implemented with or without the use of image data. For instance, the embodiments can use spike and location information. For example, with reference to FIG. 6A and the other sensor data 615 and/or the ellipsis 625, the above principles can be practiced without the use of image data. As another example, suppose image data is available. In this scenario, the embodiments can confirm the meal in the image using location and CGM spike data. Also, note that the CGM trace (or portion thereof) may be associated with the picture/meal.

Similarly, if the image data 610 (or other data, such as from wearable sensors) reflects a physical exercise or activity performed by the user, the image data 610 can be used to provide context for the user’s CGM data. If no CGM data is available during the period corresponding to the timestamp of the image, service 105 is still able to predict how those activities or food might impact the user’s glucose levels. Indeed, the embodiments are able to generate predicted analyte levels based on supplemental data, including GPS data, image data, calendar data, and so on. Thus, it should be noted how the other sensor data 615 may include any other type of data, including location data, wearable sensor data, and so forth.

Regarding the other sensor data 615, the other sensors (i.e. wearable monitors) can include any kind of step tracker, blood pressure monitor, heart rate monitor, lactate sensor, ketone sensor, alcohol sensor, glucose ketone sensor, glucose ketone alcohol sensor, glucose ketone alcohol lactate sensor, and so on. Indeed, any type of sensor can be used and can provide data. For instance, supplemental data can be fed as input to a health model or profile, and that supplemental data can be obtained from one or more of a glucose ketone sensor, a glucose ketone alcohol sensor, or a glucose ketone alcohol lactate sensor.

Regarding user input data 620, a user can provide supplemental information as to the activities he/she is engaged in and/or the food being consumed (e.g., the user may manually log exercise, insulin doses, and/or meals). A user can also provide context or information describing why his/her glucose levels are what they are.

### Software application

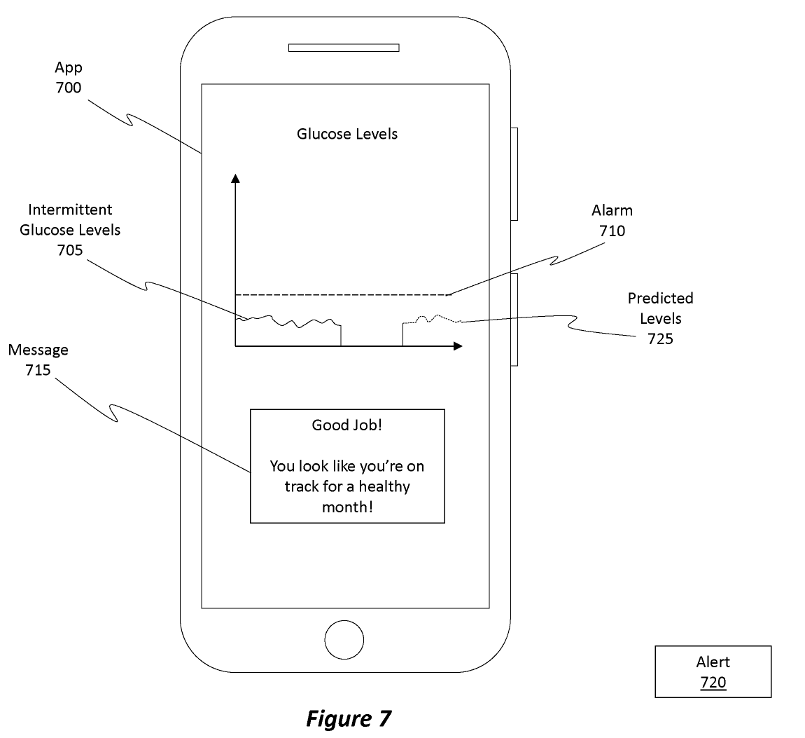

FIG. 7 shows an example app 700 that can be provided by the disclosed services. In this example scenario, app 700 shows the glucose levels for a user collected over a period of time, as reflected by the intermittent glucose levels 705. Notice, the sensor data stops at some point in, such as perhaps because the user stopped wearing the sensor. App 700 also includes an option to set an alarm 710 threshold, which can be used to inform the user if his/her glucose levels exceed the alarm 710 threshold. In other examples, an alarm threshold can be set for a low glucose event in which the user’s glucose levels fall below a low threshold, lower than alarm 710 threshold. In some examples, high and low alarm thresholds can be set.

App 700 also includes a user interface (UI) element for displaying a message 715. This message 715 can operate as an alert 720 to the user. In some cases, the alert 720 can inform the user that he/she should start wearing the sensor again in order to collect updated sensor data. In some examples, message 715 can display coaching messages or insights as described below.

Optionally, the embodiments can determine a frequency at which to transmit the alert to the user. In some scenarios, this alert can be one that recommends the user assist in obtaining new or updated sensor data. The frequency may optionally be based on one or more characteristics of the user. For instance, the characteristics can include a pattern by which the user uses the sensor, a pattern by which the user performs various activities, or a pattern by which the user consumes various foods.

App 700 also shows various predicted levels 725 for the user. The display of these predicted levels 725 may have been triggered in response to the device not receiving updated data for a determined time period. The predicted levels 725 are predicted based on the user’s historical levels and, potentially other profile information (e.g., location information, meal information, activity information). As one example, consider a scenario where a user takes a picture of the ice cream or consider a scenario where the user’s location indicates the user is physically located at an ice cream shop (in some examples, the historical data may include one or more previous glucose spikes while at this location). These supplemental forms of data can be relied on to provide an explanation as to why the glucose level spiked (e.g., the user consumed ice cream based on the picture of the ice cream and/or the user’s location).

In some implementations, the display of the predicted levels 725 is caused to be different than the display of the actual sensor data (e.g., the intermittent glucose levels 705). For instance, the predicted levels 725 can be displayed using dotted lines, different coloring, bolded lines, flashing lines, or any other visual emphasis so as to distinguish the predicted levels 725 from the actual sensor data. In some cases, app 700 may not display predicted glucose levels 725; rather, app 700 may provide insights based on the intermittent glucose levels 705.

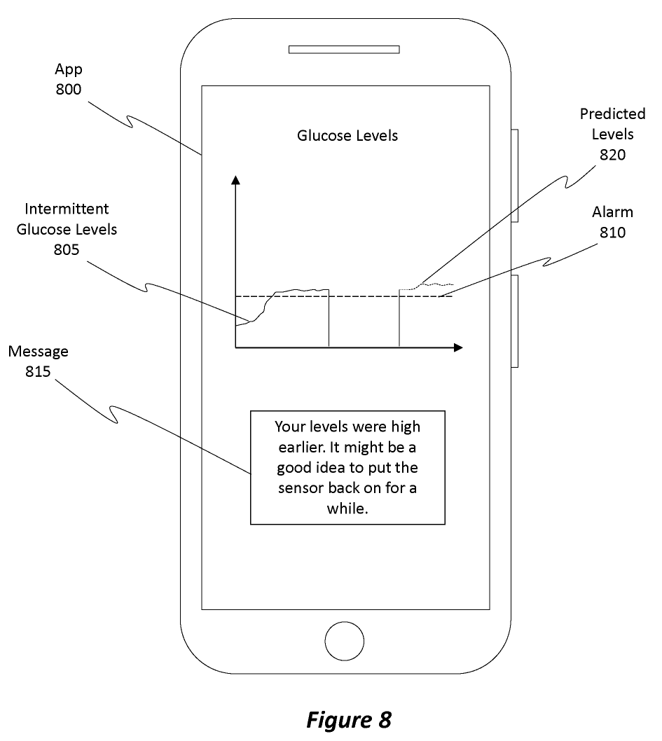

FIG. 8 illustrates an app 800 that shows intermittent glucose levels 805, an adjustable alarm 810, a message 815, and predicted levels 820. In this example, the user’s intermittent glucose levels 805 and even the predicted levels 820 are shown as exceeding the alarm 810 level. As a result, the message 815 is displaying language intended to help the user reduce his/her glucose levels by better monitoring those levels via a CGM sensor. In this example scenario, message 815 tells the user that he/she should use a CGM sensor. Different language can be used in the message 815. For instance, currently, message 815 says the following: “Your levels were high earlier.” “It might be a good idea to use a glucose sensor for a while.” In alternative scenarios, the message 815 may read: “Your glucose spiked the last time you were at this location.” “It might be a good idea to wear a glucose sensor.” In yet another scenario, message 815 may read: “Your glucose spiked the last time you ate this dish.” “It might be a good idea to have a different dish today or wear a glucose sensor.”

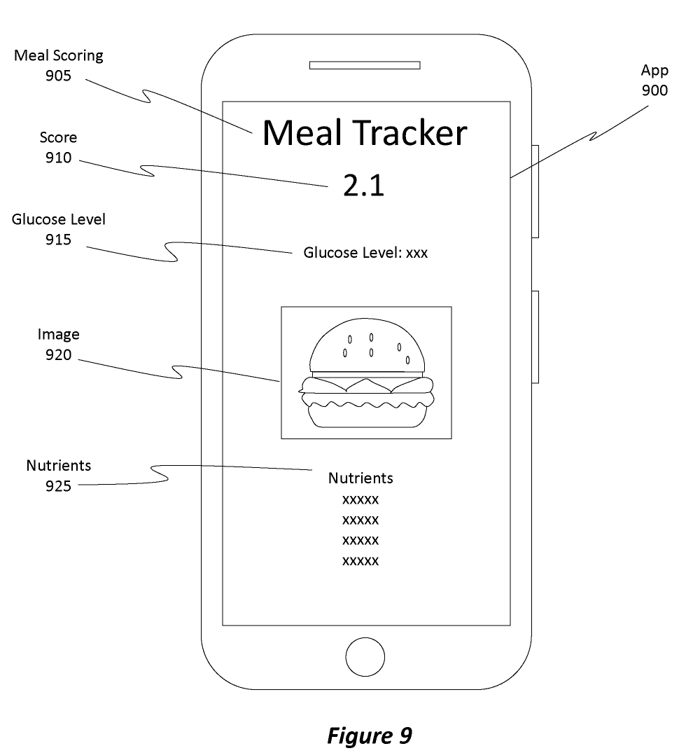

FIG. 9 shows an example app 900 that includes a meal scoring 905 feature. This feature displays a score 910 to the user, where that score 910 ranks the user’s food choices in terms of contributing to the user’s overall health (e.g., nutrition content) or, in particular, to the user’s glucose levels (e.g., carbohydrate content, glycemic index). App 900 can optionally display the user’s glucose level 915.

Different data can be used to generate the score 910. In some implementations, the user can take a picture of the food being consumed, and that picture can be uploaded to the app 900, as shown by image 920. Object recognition can be performed on the image 920 to determine what ingredients 925 are included in the food and what the nutritional details of the food are likely to be. Score 910 can be based on these estimates. In some cases, score 910 can also be based on community data, such as data from the similar user profiles.

In some cases, the user can enter input on the food. Such input can include the type of food, the portion size, the ingredients list, and so on. Input from another application can also be used. As one example, food calorie data or food macronutrient data can be used as input.

Service 105 of FIG. 1, which can implement the app 900 of FIG. 9, can be configured to include one or more user interface (UI) elements for entering eating habits of the user by tagging the glucose profile with supplement data reflective of those eating habits. Optionally, the ML model can be further trained to detect glycemic patterns of the user based on an identified order of food type intake of the user and generate recommendations on the order in which the user should consume meal components. In some scenarios, the recommended order of food type intake includes an order in which a fatty food is consumed by the user first or prior to food(s) in the picture with lower levels of fat and/or foods with higher levels of carbohydrates. In some scenarios, the recommended order of food type intake includes an order in which a food having at least a threshold level of fiber is consumer by the user first or prior to food(s) in the picture with lower levels of fiber and/or foods with higher levels of carbohydrates. In some scenarios, the recommended order of food type intake includes an order in which a food having at least a threshold level of protein is consumer by the user first or prior to foods in the picture with lower levels of protein and/or foods with higher levels of carbohydrates. These different orderings can have an impact on the user’s response to GLP-1 and/or on the user’s glucose levels. In another scenario, the embodiments may recommend that the user eat the food in a particular order, such as by eating the food in the order of fewest carbs to highest carbs. Alternatively, the embodiments can recommend that they eat the food with the highest carbs last.

In some implementations, the ML model may be trained to associate the order of food consumption within a meal with corresponding physiological responses by using training data that includes time-aligned records of meal composition, order of intake, and resulting analyte measurements. For example, a dataset may include sequences indicating the order in which different food categories (e.g., carbohydrates, proteins, fats, or fiber-rich foods) were consumed, together with corresponding glucose profiles measured during and after the meal. In some scenarios, the data may be labeled or processed to identify outcomes such as reduced postprandial glucose excursions or improved stability. The model may learn relationships between specific consumption orders and these outcomes by extracting features that encode both food type and temporal order (e.g., sequence position within a meal). During operation, when a user logs or captures information about a meal, the system may determine or infer the order of food intake and provide this information as input to the model. The model may then generate a recommendation by predicting the expected physiological response for different possible consumption orders and selecting an order associated with more favorable predicted outcomes. In some implementations, these predictions may be further refined using the user’s historical responses to similar meal compositions or intake sequences.

In some implementations, the recommendation relating to order of food intake is derived from learned associations between intra-meal sequencing and subsequent physiological responses. This enables the system to generate recommendations that modify the sequence of consumption within a single meal, which differs from approaches that only evaluate aggregate nutritional content or total intake.

Service 105 can also provide a user interface element in app 900 to enter mealtimes of the user. Optionally, service 105 can automatically detect mealtimes based on changes to a user’s real-time glucose levels. This detection can also occur based on recognized eating patterns of the user, such as based on timing data. For instance, service 105 can detect that the user routinely eats a meal at around a certain time of day.

Optionally, service 105 can include a first option (perhaps in app 900) for the user to enter a workout performed by the user. A second option can be provided to tag the user’s glucose profile based on a timing of the workout. Other activities can be tagged as well, without limit.

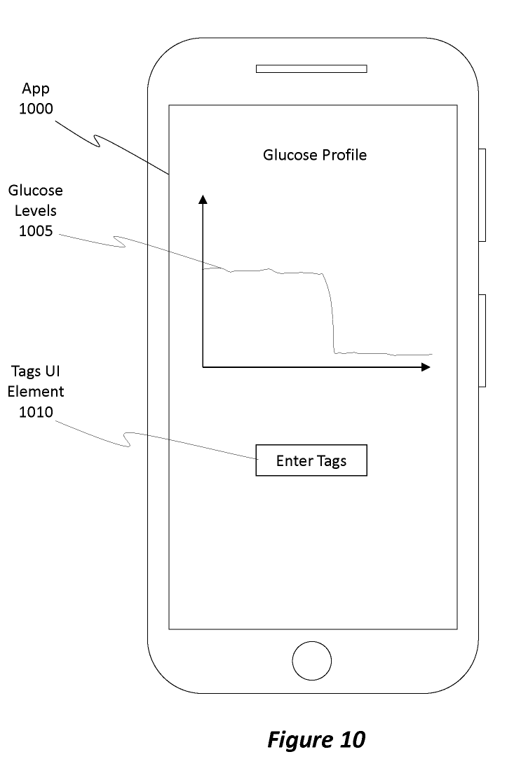

FIG. 10 shows an app 1000 that is representative of the apps mentioned thus far. App 1000 is shown as displaying the user’s glucose levels 1005. Notice, there is a significant change in the pattern of the user’s glucose levels. This significant change may occur due to the introduction of a new pharmaceutical, such as the GLP-1 540 or GLP-1 + GIP 545 described in FIG. 5A. Thus, the pattern change in FIG. 10 can correspond to the pattern change described in FIG. 5A. App 1000 can include an option (e.g., tags UI element 1010) to allow the user to tag specific data with additional information to color or provide additional insight as a reason for that data.

### GLP-1 RA dosage recommendation method

The following discussion now refers to a number of methods and method acts that may be performed. Although the method acts may be discussed in a certain order or illustrated in a flow chart as occurring in a particular order, no particular ordering is required unless specifically stated, or required because an act is dependent on another act being completed prior to the act being performed.

Attention will now be directed to FIG. 11A, which illustrates a flowchart of an example method 1100 for using a machine learning (ML) model to recommend continuance or modification of a usage of a glucagon-like peptide-1 (GLP-1) treatment of a user, where the GLP-1 treatment is designed to treat a physiological condition of the user, and where one or more continuous glucose monitors (CGMs) monitor glucose levels of the user. Notably, the monitored glucose levels are used to facilitate the ML model’s recommendation regarding continuance or modification of the GLP-1 treatment. Method 1100 can be implemented within architecture 100 of FIG. 1. Also, method 1100 can be performed by service 105.

Method 1100 includes an act (act 1105) of configuring a service associated with the one or more CGMs to operate in a blinded mode (or diagnostic mode) during a first period of time in which the one or more CGMs measure the glucose levels in a body of the user. The service, when operating in the blinded mode, prevents or eschews display of measurements obtained by the one or more CGMs during the first period of time. The measured glucose levels obtained during the first period of time are included in a glucose profile for the user.

Alternatively, the service can be caused to operate in a non-blinded mode during the first period of time. While operating in the non-blinded mode, the service can display the measurements obtained by the CGMs during the first time period.

Configuring the service to operate in the blinded mode can be beneficial for various reasons, as previously mentioned. One primary reason for hiding data from a user is that the user will often modify his behavior if his metrics are made readily available. For instance, if a user has access to data and sees certain spikes or dips after certain activities, the user may refrain from performing those activities. If the user is not made aware of the data, however, then the likelihood of behavior modification is reduced. Thus, operating in the blinded mode for a period of time allows the embodiments to obtain a baseline set of accurate (or likely accurate) data reflective of the user’s behavior.

Subsequent to the first period of time, act 1110 includes configuring the service to operate in a non-blinded mode during a second period of time in which the one or more CGMs measure the glucose levels in the user’s body. The service, when operating in the non-blinded mode, enables display of measurements obtained by the one or more CGMs during the second period of time. The measured glucose levels obtained during the second period of time are included in the glucose profile for the user.

As mentioned earlier, the service can initially be caused to operate in the non-blinded mode. If that is the case, then the first and second periods of time can effectively be merged into the same period of time.

Operating in the non-blinded mode during the second period of time is also beneficial for various reasons. One reason is to allow the ML engine and service to acquire additional baseline data. Another reason is to determine whether the user does change his/her behavior when presented with the CGM data.

In some implementations, the ML engine that generates recommendations regarding continuation or modification of a GLP-1 treatment may be trained using datasets that include historical physiological data in combination with corresponding therapy decisions and outcomes. For example, training data may include glucose profiles collected over defined periods (e.g., multiple days or weeks with periodic measurements), together with records indicating when GLP-1 treatments were initiated, continued, adjusted, or discontinued, and any associated clinical outcomes (e.g., changes in glucose control, weight, or adverse events). In some scenarios, the training data may further include clinician decisions or documented treatment adjustments, which may be used as labels indicating appropriate actions under given conditions. The model architecture may include one or more components adapted to process temporal physiological data (e.g., sequences of glucose measurements) alongside contextual inputs such as medication information or patient characteristics, with these inputs combined to produce an output indicative of a recommended action (e.g., maintain, increase, decrease, or modify therapy). During training, the model may learn associations between observed patterns in the input data and corresponding therapy decisions or outcomes, for example by minimizing a loss function that reflects agreement between predicted and observed actions or results. During operation, the ML engine may apply these learned associations to current user data to generate a recommendation, optionally incorporating both population-level patterns and user-specific historical data.

A few optional acts are illustrated in FIG. 11B. For instance, act 1110A includes determining that the one or more CGMs were used intermittently at least during one of the first or second periods of time. Act 1110B then includes causing the ML model to generate predicted glucose levels during time periods when the one or more CGMs were used intermittently. This predicted data can be used to augment, supplement, or fill in the data that was missing.

Returning to FIG. 11A, act 1115 includes determining that the user has initiated the GLP-1 treatment. The GLP-1 treatment involves dosing the user using a first dosage level of a GLP-1. The initiation of the GLP-1 treatment can, in some instances, occur subsequent in time to either one of the first or second periods of time. Preferably, the treatment commences after the second period of time. In some scenarios, the GLP-1 treatment and the monitoring of the user’s glucose levels start at the same time or substantially at the same time.

Act 1120 includes adding tagged data to the glucose profile for the user. The tagged data includes a timestamp reflective of when the user initiated the GLP-1 treatment. The tagged data can also include data reflective of the first dosage level.

During a third period of time subsequent to the second period of time, act 1125 includes using the one or more CGMs to measure the glucose levels of the user’s body while the user is taking the GLP-1 (e.g., on a GLP-1 medication cycle). The measured glucose levels obtained during the third period of time are included in the glucose profile for the user.

In response to the ML model detecting a change to a glycemic pattern (e.g., glycemic variability) of the user during the third period of time (as compared to the patterns that might exist during the first or second periods of time), act 1130 includes causing the ML model to generate a recommendation for the user. In some implementations, the recommendation is not determined by evaluating whether a measured value satisfies a predefined threshold condition. Instead, the ML model may generate outputs based on learned relationships across multiple features, such that similar measured values may result in different recommendations depending on context. This enables more flexible and individualized decision-making compared to rule-based threshold systems.

This recommendation is that the user should have a particular dosage level (e.g., maintain the current dosage level or use a different and new dosage level) of the GLP-1 based on the detected change to the glycemic pattern and optionally based on the predicted glucose levels generated by the ML engine. Thus, the recommendation can indicate that the current dosage level is to be maintained or can indicate that the current dosage level is to change (e.g., either be increased or decreased). In one example, an increase can be recommended if GMI, A1C, or glucose variability is above a threshold. Alternatively, the recommendation may include down titrating if below a threshold (e.g., below 7% for GMI or A1C). In other examples, the titration determination can include weight as input. Some scenarios involve a situation where the user’s goal is weight loss. The user/HCP may want to up titrate because the user is not losing weight or not losing weight fast enough, and the ML model and CGM data can be used to determine whether an up titration would not be recommended or can be used to determine the amount to up titrate.

In some implementations, the detection of glycemic patterns and the generation of recommendations based on those patterns may be performed by a single ML model or by multiple coordinated components within a unified model framework. For example, a first portion of the model may be trained to process time-series physiological data and generate intermediate representations or outputs indicative of detected patterns (e.g., trends, variability, or recurring excursions), while a second portion of the model may use those intermediate representations, optionally together with additional inputs (e.g., medication data or user characteristics), to generate a recommendation. In some scenarios, these portions may be implemented as distinct stages within a single model architecture (e.g., shared feature extraction layers followed by task-specific output layers), while in other scenarios the tasks may be performed by separate models that exchange outputs. During training, the model or models may be trained jointly or sequentially using datasets that include both physiological data and corresponding outcomes or decisions, thereby enabling the system to learn both how to identify relevant patterns and how those patterns relate to appropriate therapy recommendations.

The measured glucose levels obtained during the different periods of time are used by the ML model to generate the recommendation. For instance, the ML model is trained to identify patterns in data. By providing the measured glucose levels to the ML model, the ML model can implemented its training to detect patterns in the measured glucose levels. It should be noted how the disclosed ML model can be a single ML model as opposed to the combination of multiple different ML models. That is, the ML model can be trained to perform numerous different actions, as recited herein. Also, the disclosed embodiments collect various additional data and provide that data to the ML model to help the ML model generate its recommendation and to help the ML model perform its other operations. Regarding the time periods, one example reason as to why these times periods are explicitly defined and separated out from one another is to provide a baseline amount of data to the ML model to detect changes in the user’s glycemic profile. As discussed earlier, the first and second periods of time can operate as seed data or baseline data to assist in detecting the normal physiological behavior of the user. This baseline data can then be compared against data collected while the user is undergoing the GLP-1 treatment.

In addition to being implemented as a single model or as multiple coordinated models, the ML model may include components that operate at different stages of processing. For instance, one component may process time-series analyte data, while another component processes supplemental data such as dietary inputs or activity data. These components may operate sequentially, in parallel, or in a hybrid arrangement, and their outputs may be combined to generate a unified recommendation. In some cases, one or more components may be rule-based or heuristic-driven, while other components rely on learned representations.

In some implementations, one or more portions of the described functionality may be performed using deterministic logic, heuristic rules, statistical models, or combinations thereof, either in place of or in conjunction with machine learning techniques. For example, predefined rules based on observed glucose patterns or threshold conditions may be used to generate or refine recommendations. In such cases, the system may operate without requiring a trained machine learning model.

Prior to detecting the change, the ML model is applied to the user’s data to detect the patterns and changes in patterns that were previously present in the user’s data. The ML model can access any of the input data mentioned previously to determine how to modify the user’s dosage level. Subsequently, the service can continue to monitor the user’s CGM data during different periods of time.

The ML model can be further trained (it was previously trained) based on updated training data. The ML model can also be further tuned. In response to this additional training, the ML model can subsequently be applied to the user’s data to generate yet another recommendation or any number of subsequent recommendations. In some cases, real-time recommendations can be generated as new user sensor data (or other data, such as user input, image data, location, historic data, etc.) is received. Additionally, or alternatively, the ML model may continuously, at least for a period of time, monitor the user’s reading while dosing at the new level.

In other implementations, recommendations may be generated on a deferred or scheduled basis rather than in real time. For example, the system may aggregate data over a defined period and generate recommendations at periodic intervals, such as after sufficient data has been collected to identify a meaningful pattern. This approach can reduce sensitivity to short-term fluctuations and provide more stable guidance, particularly in scenarios where data is intermittently available or where longer-term trends are of interest.

In some scenarios, causing the ML model to generate a recommendation for the user involves recommending that the user either titrate up or titrate down the GLP-1 based on the detected change to the glycemic pattern and/or weight loss. Optionally, the recommendation can include a specific titration amount for the user.

Optionally, the recommendation can include a recommendation to switch from dosing the GLP-1 to dosing a dual GLP-1 and a gastric inhibitory polypeptide (GIP) drug. The switch may be triggered based on a predetermined factor (e.g., when a maximum GLP-1 dose has been reached). The recommendation can also include other dosages, titration amounts, titration rates of change, and so on.

In some scenarios, data can be collected during other time periods as well. For instance, a CGM can be used to collect data while no GLP-1 is being used by the user. Another time period may occur where the user is dosing GLP-1 but no CGMs are being used to monitor the user’s levels. A CGM may be in use during a time when a new cycle or dosage of a GLP-1 is being used.

In some scenarios, the recommendation indicates that the particular dosage level of the GLP-1 is to be an increased titration of the GLP-1. The increment can be a predetermined increment or a dynamic increment. For instance, the predetermined increment can optionally be some predetermined percentage or absolute value amount increase. If dynamic, the increment can be based on the current, past, or a combination of current and past CGM data or other data. In other scenarios, the recommendation indicates that the particular dosage level of the GLP-1 is to be a decreased titration of the GLP-1 or, alternatively, an adjustment to an insulin level. This reduction can be based on the same factors described above in connection with the increase.

In some implementations, the ML model further relies on a GMI value or a glucose pattern insights (GPI) report to generate the recommendation. For instance, the GMI value or GPI report 640 from FIG. 6B can be used to generate the recommendation.

In some implementations, the ML model is further trained to predict whether the user has prediabetes based on the glucose profile. Note, in one example, a GMI value above a threshold value (e.g., about 5.5%) can result in a recommendation to obtain an A1C result and/or to see an HCP. The ML model can also optionally detect that an appetite of the user is likely reduced subsequent to treatment using the GLP-1 based on a detected change to the glycemic pattern, which is reflected in the glucose profile.

In some implementations, the ML model may be trained to predict prediabetes using datasets that include glucose profiles collected over defined monitoring periods together with corresponding diagnostic labels. For example, training data may include glucose measurements obtained at periodic intervals over multiple days or weeks, along with associated clinical determinations such as normoglycemic, prediabetic, or diabetic classifications based on established diagnostic criteria (e.g., laboratory measurements or clinician diagnoses). In some scenarios, the glucose profiles may be processed to extract features such as average glucose levels, variability, time in range, or patterns of excursions, and these features may be provided as input to the model. The model architecture may include one or more components adapted to process time-series data or derived features and produce an output indicative of a predicted classification or likelihood. During training, the model may learn relationships between characteristics of the glucose profile and the corresponding diagnostic labels, for example by optimizing a loss function that reflects agreement between predicted and actual classifications. During operation, the trained model may apply these learned relationships to a user’s glucose profile to generate a prediction regarding prediabetes, optionally in combination with other available data.

As another option, an insulin dispersal rate of an insulin pump of the user can be modified based on the glucose profile and based on the recommendation generated by the ML model. Thus, the insulin pump can be dynamically (e.g., in real time) controlled based on the data provided to the ML model. In another example, insulin doses with an insulin pen can be titrated (for basal and/or prandial insulin). After dosing the GLP-1, the ML model can detect a new pattern of insulin injection provided by an insulin pump used by the user. In some scenarios, a second dosing of the GLP-1 can be tagged and recorded using the service.

Optionally, the ML model can be a supervised ML model. Patterns and weight loss can be tagged and used by the supervised ML model. Optionally, the ML model can be unsupervised.

Method 1100 can also include an act of generating a glucose pattern insights (GPI) report. This report includes patterns of glucose behavior for the user. The ML model can determine a treatment protocol based on the patterns in the GPI report.

In some implementations, the ML model predicts that the user should titrate up in dosing the GLP-1. The service can display a predicted titration rate generated by the ML model. Alternatively, the ML model can predict that the user should titrate down in dosing the GLP-1, and the service can display a predicted titration rate generated by the ML model.

In some implementations, the ML model includes generative artificial intelligence (AI). The generative AI can be used to capture an image of food consumed by the user, and the generative AI can determine a nutrient value of the food based on the image. Optionally, the generative AI can determine a food pattern of the user. Food pattern can be any detected consumption of food for a user. For instance, food pattern may be a diet pattern or a typical glucose response to a particular food. Optionally, the generative AI can determine whether a food pattern of the user has changed over time while the user is dosing the GLP-1. Changes in the user’s insulin response pattern can also be detected, particularly since starting the GLP-1. In some implementations, the service captures the image and provides the image to the generative AI. Thus, in some scenarios, the generative AI avoids capturing the image and leaves that task to the service.

The service can include an option to track a nausea level of the user while the user is dosing the GLP-1. The service can include an option to track one or more of a sleep rate, a heartrate, a medication consumption rate, or a bowel movement rate of the user while the user is dosing the GLP-1. Note, nausea information can be obtained by allowing the user to log nausea episodes and/or by surveying users about their nausea. The embodiments can also detect a missed meal and can ask the user to confirm whether he or she is not hungry and/or nauseous (e.g., in an alert or prompt).

The service can communicate with a wearable device worn by the user. The wearable device detects activities of the user, and the service receives data reflective of the activities. Optionally, the service tags the glucose profile with the data reflective of the activities.

In some scenarios, the one or more CGMs are used intermittently at least during one of the first period of time or the second period of time. The ML model can be further configured to predict glucose levels during the intermittent use of the one or more CGMs. The ML model can also generate the recommendation based, at least in part, on the predicted glucose levels.

Typically, a GLP-1 will delay the need for insulin for diabetics; thus, the embodiments can detect when an insulin response pattern has changed for the user, particularly since starting a GLP-1 treatment. The embodiments are able to track the user’s glucose trends and help diabetics determine a proper treatment protocol. The embodiments can also use glucose sensors intermittingly to determine when a GLP-1 treatment is no longer working. Thus, the embodiments can determine when to escalate therapy and/or when to de-escalate a therapy. Different triggers may be provided for identifying when a GLP-1 treatment is no longer working. Example triggers include, but are not limited to, a scenario where GMI is rising over time, glucose variability is rising over time, or A1C is rising over time. In the scenario where the user is cycling on GLP-1, this could mean that the GLP-1 does not have the same effect on GMI, glucose variability, and/or A1C as it did before.

The embodiments can also infer whether a user is experiencing nausea from dosing the GLP-1 based on whether a user is skipping meals. Often, a skipped meal is reflective of an underlying problem, such as nausea. Thus, the embodiments can discern whether the user is having nausea based on that user’s glucose profile, which will reflect whether a meal has been skipped. If a user is skipping meals, it might be the case then that the user is not obtaining a sufficient amount of nutrients. Thus, the ML generated recommendation can include guidance on a nutrient-rich diet and/or can advertise nutrition products. In some examples, the ML generated recommendation can include coaching or education on how to reduce nausea.

Some embodiments determine that the user has initiated the GLP-1 treatment. The GLP-1 treatment involves dosing the user using a first dosage level of a GLP-1. The embodiments can add tagged data to a glucose profile for the user. The tagged data includes a timestamp reflective of when the user initiated the GLP-1 treatment and includes data reflective of the first dosage level.

The embodiments use the one or more CGMs to measure glucose levels of a body of the user while the user is dosing the GLP-1. The measured glucose levels are included in the glucose profile for the user.

The embodiments cause the ML model to detect a glycemic pattern of the user. In response, the embodiments cause the ML model to generate a recommendation for the user to have a particular dosage level of the GLP-1 based on the detected glycemic pattern.

As mentioned earlier, at least some of the disclosed embodiments address both physician-specific and patient-specific challenges in GLP-1 RA therapy by leveraging data from CGMs, CKMs, CLMs, continuous alcohol monitor, continuous multi-analyte monitors, and dietary information from food apps.

Beneficially, the embodiments can optimize therapy by using metrics derived from the continuous analyte data (e.g., CGM data). The embodiments can inform concurrent therapy adjustments, such as insulin titration, and can guide GLP-1 RA titration.

The embodiments also beneficially provide early response detection. For example, early changes in CGM-derived metrics (e.g., time in range (TIR), GMI, etc. over first few sensor wears) can help identify responders vs. non-responders in terms of glucose control and weight loss and other clinical benefits, thereby enabling physicians to tailor treatment more effectively, minimize unnecessary exposure to side effects for non-responders, and minimize unnecessary healthcare expenditure.

The embodiments can also facilitate nutritional monitoring. Here, integrating CGM data with CKM data and dietary intake data from food tracking apps can provide a comprehensive view of the patient’s nutritional pattern during the GLP-1RA therapy, thereby enabling close monitoring and timely interventions, particularly in cases where patients may be skipping meals or lacking adequate nutritional intake.

The embodiments can also facilitate adherence detection. Trends in CGM-derived metrics like time in range (TIR), time above range (TAR), fasting glucose (FG)can help detect suboptimal GLP-1 RA adherence.

Some embodiments can also trigger ketone based alerts. For instance, rising ketone levels or prolonged elevated ketone levels detected via CKM can be used to trigger meal reminders or alerts to prevent malnutrition and ketoacidosis in GLP-1 RA users.

Some embodiments are also structure to provide motivational feedback. Here, CGM data can be used to display improvements in glucose control, thereby reinforcing adherence to GLP-1 RA therapy through personalized feedback and progress tracking.

Some embodiments can also perform alcohol intake monitoring. For instance, continuous alcohol monitoring can help reduce GI discomfort and can support adherence by providing alerts, behavioral insights, and positive reinforcement for reduced alcohol consumption.

Some embodiments can also help with preserving muscle mass. For instance, tracking lactate levels during anaerobic exercise helps ensure sufficient intensity to preserve muscle mass and to prevent overtraining in individuals on GLP-1 RAs.

### Insulin dosage recommendation method

With the above in mind, this disclosure will now describe various embodiments that use continuous glucose, ketone, lactate, and/or alcohol monitoring data, along with dietary data from food apps to address challenges associated with GLP-1 RA treatment. Various features of the embodiments include optimizing treatment adjustments, early response detection, adherence monitoring, nutritional assessment, timely alerting, and motivational feedback to mitigate therapy discontinuation. Some implementations can be divided into two parts, with one part being physician facing features and the other part being patient facing features.

### Physician Facing Features

GLP-1 RA titration: GLP-1 RA doses can be adjusted based on specific medication, patient tolerance, and therapeutic goals (e.g., glycemic control or weight loss). In general, these medications are started at a low dose and are adjusted slowly to reduce GI side effects. Doses are typically titrated every 4 weeks (or weekly for liraglutide/Saxenda) depending on the drug. In obesity management, dose escalation is often continued to the maximum tolerated dose to optimize weight loss. For patients with T2D however, the guidelines suggest dose escalation if HbA1c remains above target, and/or fasting or post-prandial glucose levels are not at goal. CGM can support these decisions by providing actionable metrics, such as time in range (TIR), time above range (TAR), fasting glucose (FG), glucose variability metrics like Mean Amplitude of Glycemic Excursions (MAGE), postprandial Area Under the Curve (pp-AUC) to track postprandial excursions, Glucose Management Indicator (GMI), and so on.

One or more of these metrics can be monitored during a predefined period (e.g., 4 weeks) with a pre-specified threshold for change (e.g., a 5% improvement in TIR). If insufficient improvement is observed and glucose control is not at target, this can indicate non-adherence and/or inadequate dose. In such cases, a notification can be sent to the physician to prompt dose reassessment or patient consultation.

Concurrent insulin dose adjustment: GLP-1 RAs enhance glucose-dependent insulin secretion, suppress glucagon secretion, and delay gastric emptying. When combined with exogeneous insulin in patients on long-acting insulin (LAI) therapy or multiple-daily injections (MDI), these effects can amplify glucose lowering, potentially leading to hypoglycemia when insulin doses are not adjusted. This can be addressed by using CGM data to predict risk of hypoglycemia with GLP-1 RA initiation or escalation in insulin users.

Initiating GLP-1 receptor agonists (GLP-1 RAs) in insulin-treated diabetes patients can increase hypoglycemia. To combat hypoglycemia, insulin reduction may be recommended for patients with diabetes with HbA1c below a threshold (e.g., HbA1c <8%), but this would not account for glucose variability captured by continuous glucose monitors (CGM). A retrospective analysis was conducted using linked CGM data from FreeStyle Libre (FSL) devices and insurance claims. This analysis explored the use of CGM data before GLP-1 RA initiation to identify patients that would need insulin reduction. Using CGM data from linked FreeStyle Libre 2 (FSL2) and medication claims, the results identified insulin-treated patients initiating or re-initiating GLP-1 RAs. Three binary classifiers were developed based on CGM data within the 30 days before initiation: (1) GMI-only, (2) time below 70 mg/dL (TB70)-only, and (3) a multi-feature logistic regression model incorporating TB70, median glucose, and glucose variability. The classifiers aimed to detect patients with TB70 >= 2% in the 30 days following GLP-1 RA initiation, indicating a need for insulin reduction.

FIG. 12A shows a receiver operating characteristic curve for predicting 1052 patients needing insulin reduction from a cohort of 4384 with GMI and TB70 thresholds. The GMI model had the lowest performance (AUC = 0.65). TB70 and the multi-feature model performed significantly better (AUC = 0.86 and 0.87, respectively). For similar sensitivity, the TB70 and multi-feature models detected 700 and 633 false positives, compared to 2032 with GMI <8% (surrogate for HbA1c). CGM data before initiation helps identify patients likely to experience hypoglycemia post-initiation, enabling preemptive insulin reduction. Reducing false positive prediction prevents unnecessary insulin reductions preserving the therapeutic benefits of GLP-1 RAs.

The disclosed embodiments provide various techniques to enable recommending individualized insulin dose reductions prior to the initiation of GLP-1 RA therapy in insulin using patients with diabetes, with the objective of minimizing hypoglycemia risk after initiation of the GLP-1 RA therapy. In some implementations, the determination of an insulin dose adjustment prior to initiation of the therapy is based on predicted physiological responses derived from pre-initiation data rather than observed responses during therapy. For example, the ML model may evaluate features extracted from baseline analyte data (e.g., frequency, duration, or severity of low-glucose events, variability metrics, or temporal patterns) to estimate a likelihood of hypoglycemia under a projected therapy condition. The recommendation may then be generated based on this predicted likelihood rather than on observed post-initiation outcomes, thereby enabling prospective adjustment of therapy parameters prior to exposure.

The embodiments utilize a machine learning model trained on multimodal data sources, including electronic health records (EHR), CGM metrics, and clinical study datasets capturing insulin dosing patterns before and after GLP-1 RA initiation. Input features include patient demographics, current insulin regimen (i.e. dosage), GLP-1 RA type and dosage, medical history, and diabetes-related laboratory values. The embodiments can output a recommended insulin dose adjustment, either maintaining the current dose or suggesting a reduction, based on the predicted likelihood of hypoglycemia. The embodiments can be integrated into clinical decision support tools, such as a GPI report, and can be provided to clinicians as a feature for insulin-using patients if they are being initiated on GLP-1 RA.

In some implementations, clinician interaction with the system may include reviewing, modifying, or annotating the generated recommendations. For example, a clinician may adjust a recommended dosage change, provide feedback indicating whether a recommendation was accepted or overridden, or supply additional contextual information. Such interactions may be recorded and used to further refine subsequent recommendations or to provide additional context for future analyses. This interaction can occur through a clinical interface, a report, or another suitable mechanism.

For example, the dose of long-acting insulin may be reduced by approximately 10–20% in patients at elevated risk of hypoglycemia. For individuals using both long-acting and rapid-acting insulin, mealtime insulin doses may be reduced by 25–40%. These adjustments are based on expert recommendations. The extent of insulin dose reduction may also be influenced by the presence and pattern of existing hypoglycemia. Greater reductions may be warranted when hypoglycemia is already present. For example, if hypoglycemia occurs predominantly at night, a more substantial reduction in basal insulin may be appropriate. Similarly, if postprandial hypoglycemia is observed following specific meals, corresponding mealtime insulin doses may be selectively reduced to mitigate risk. These patterns of hypoglycemia can be identified through tools such as a glucose pattern analysis or other metrics, including Time Below 70 mg/dL (TB70).

The specific magnitude of any therapy adjustment may vary widely depending on patient-specific factors, model outputs, or clinical considerations. Accordingly, the described adjustment ranges are illustrative examples and are not required for all implementations. In some scenarios, adjustments may be determined dynamically without reference to predefined ranges.

In some embodiments, the degree of insulin dose reduction may be guided by the predicted risk of hypoglycemia. Specifically, smaller reductions may be appropriate when the predicted risk is relatively low, whereas larger reductions may be warranted in the context of higher predicted risk. This risk-based approach allows for more individualized insulin titration, balancing glycemic control with safety.

In some embodiments, the extent of insulin dose reduction may depend on the specific GLP-1 RA being initiated (i.e. the GLP-1 RA type). Long-acting GLP-1 RAs, such as liraglutide and dulaglutide, lower postprandial glucose excursions across all meals of the day and have a pronounced impact on fasting glucose levels. In such cases, reductions in both long-acting and rapid-acting insulin may be appropriate. In contrast, short-acting GLP-1 RAs, such as lixisenatide, exert a more targeted effect on postprandial glucose, primarily for meals consumed shortly after administration. Therefore, in these cases, a reduction in mealtime insulin may be prioritized, while adjustments to long-acting insulin may be minimal or unnecessary.

Embodiments of this disclosure can simplify patient’s medication regimen. Because GLP-1 RAs enhance insulin secretion, suppress glucagon secretion, and enhance insulin sensitivity, glucose lowering agents that also carry a higher risk of hypoglycemia such as Meglitinides (Meg) and Sulfonylureas (SU) can be removed from some patient’s regimens when initiating GLP-1 RAs. CGM data can be used to detect nocturnal or unexplained hypoglycemia in patients taking Meg and/or SU. If such episodes are detected (or if a threshold number of episodes are detected within a predefined period of time), Megs and/or SU can be discontinued as GLP-1 RA is initiated. In addition, postprandial excursion metrics such as MAGE and pp-AUC can be used to monitor postprandial excursions to escalate GLP-1 RA as Meg and/or SU are gradually deprescribed.

In some implementations, postprandial excursion metrics may be used to guide coordinated adjustment of therapies as Meglitinides (Meg) and/or Sulfonylureas (SU) are reduced or discontinued. For example, metrics such as Mean Amplitude of Glycemic Excursions (MAGE) and postprandial area under the curve (pp-AUC) may be calculated from analyte data to quantify the magnitude and cumulative exposure of post-meal glucose excursions. As Meg and/or SU are gradually deprescribed, the system may monitor changes in these metrics to evaluate whether postprandial glucose control is maintained, improved, or degraded. In scenarios where postprandial excursions remain stable or improve despite reduction of Meg and/or SU, the system may generate a recommendation to escalate a GLP-1 RA dosage. Conversely, if the metrics indicate increased variability or elevated postprandial exposure, the system may adjust or defer escalation and/or generate alternative recommendations, such as modifying dietary inputs or adjusting other therapies. In this manner, postprandial excursion metrics can be used as part of a multi-factor evaluation to dynamically coordinate therapy transitions.

In some implementations, escalation of a GLP-1 RA may be determined based on changes in postprandial excursion metrics over time as Meglitinides (Meg) and/or Sulfonylureas (SU) are reduced or discontinued. For example, the system may determine a reference level for one or more postprandial excursion metrics, such as Mean Amplitude of Glycemic Excursions (MAGE) and postprandial area under the curve (pp-AUC), and may subsequently recalculate those metrics during later monitoring periods as Meg and/or SU are gradually deprescribed. The system may compare the later-calculated metrics to the reference level and/or to metrics from one or more intervening periods to determine whether postprandial glucose control is being maintained, improved, or degraded. Where the comparison indicates that postprandial control is maintained or improved notwithstanding reduction of Meg and/or SU, the system may determine that escalation of the GLP-1 RA is appropriate. Where the comparison indicates worsening postprandial excursions, the system may defer escalation, reduce a rate of escalation, or generate a different therapy recommendation. In this manner, escalation may be calculated based on longitudinal changes in postprandial excursion behavior rather than based on a single glucose value in isolation.

Embodiments of this disclosure can identify early responders to GLP-1 RA for weight management. Changes in CGM-derived metrics over one or two sensor wears (typically 14–28 days) can offer early insights into how a patient is responding to GLP-1 RA therapy for weight management, even before significant weight loss is observed. GLP-1 RAs influence glucose metabolism and appetite regulation, and these effects can manifest in the CGM data early on. Improved satiety and reduced caloric intake may be indicated by fewer and smaller postprandial glucose excursions identified in CGM data. Changes in post-prandial excursions can be quantified using CGM-derived metrics such as the MAGE and pp-AUC. A reduction in CGM-metrics and the frequency of postprandial excursions during the first one or two sensor wear periods (e.g., 14-30 days wear) may serve as a proxy for improved metabolic control from the therapy. By systematically analyzing changes in these metrics and frequency of postprandial excursions, predictive models can be developed to identify early responders to GLP-1 RA therapy in the context of eight management. In some examples, insights from the model can be included in a CGM report for the clinician.

Embodiments of this disclosure can assess GLP-1 RA adherence.: Adherence to GLP-1 RA therapy is a widely recognized issue, particularly due to factors like gastrointestinal (GI) side effects, cost, and delayed perceived benefits. In patients with T2D, CGM can reflect glucose lowering effects of GLP-1 RA. Expected glycemic improvements may not be observed when adherence is poor or inconsistent. Lack of improvement in CGM-derived features like TIR, TAR, FG, GMI, MAGE, pp-AUC, etc., over one or two sensor wears (e.g., 14-30 days wear) may indicate non/low-adherence where lack of improvement is defined as a change in these metrics that falls below a predefined threshold. In addition, reversal or plateau in metrics such as TIR, TAR, which may be visible in weekly patterns, can indicate missed doses. Metrics such as TIR, TAR, and FG can be calculated on a daily basis, and their temporal trends may offer insights into the frequency of GLP-1 RA administration.

For instance, when taken as per the prescribed frequency, GLP-1 RA maintains a constant concentration in the body, but missed doses can lead to declining levels in the body. As the concentration starts to deplete resulting in diminished efficacy, metrics like daily Time Above 180 mg/dL (TA180) may start to rise. Such patterns in daily TA180 can help estimate whether a patient is taking their dose weekly, every 10 days, or with longer intervals between doses. Such insights can be included in a CGM report. Finally, physicians can use this adherence information to make more informed therapy adjustments (e.g., GLP-1 RA, insulin).

Embodiments of this disclosure can monitor food portions, macro-nutrient intake. For example, dietary data from food apps can be used in conjunction with the CGM data to monitor food portions and macronutrient (carbohydrates, proteins, fats) intake. Information about portion sizes, macronutrient composition and meal timing in conjunction with the CGM data can be used to assess how specific foods, macronutrients ratios, or meal patterns (e.g., order of food intake, meal frequency, meal times) influence glucose excursions with the GLP-1 RA therapy which can guide future food choices.

For example, consuming a high GI food like white bread can cause a sharp postprandial glucose rise in patients who have more impaired insulin secretion or fast gastric emptying. Personalized recommendation for this patient can include suggesting lower-GI options with more fiber and protein such as lentils. The same meal can show a modest increase in postprandial glucose for another patient, but the patient may be more sensitive to food portions. Personalized guidance can include focus on food portion and consistent meal timing for this patient. Similarly, for patients with high-sensitivity to carb-first meals, suggestion can include eating vegetables and protein first and delaying carbohydrate intake. Such insights can be presented to the user or an HCP in a CGM app or a report.

In addition, users can log food that cause nausea or log symptoms and severity to identify food that cause nausea. This information can be used to identify food such as high fat meals, spicy foods, or portion sizes that trigger GI discomfort with the GLP-1 RA therapy. Such insights can be included in a report or app for both user and clinician facing use. Dietary adjustments based on identified triggers can help improve overall tolerability of the therapy. Furthermore, food logs or captured food pictures can be used along with the CGM data to identify skipped meals or prolonged fasting periods. For example, absence of glucose excursion during expected mealtimes, alone or in combination with the absence of logged food intake or food picture, can indicate skipped meal. This can be relevant to monitor or capture malnutrition and can be added to a clinician facing report to facilitate a discussion with the patient.

### Patient Facing Features

Embodiments of this disclosure can provide eating reminders based on ketone levels. GLP-1 RA slows gastric emptying, enhances satiety, and decreases food cravings. This results in reduction in food intake, potentially skipped meals, and prolonged fasting periods in GLP-1 RA users. This can induce fasting like metabolic state leading to increased fat metabolism and ketone production. While this can support weight loss, it can also lead to malnutrition and ketoacidosis. High or rising ketone levels, above a predetermined threshold sustained over time, can indicate insufficient food intake. This concept can be implemented as a feature in the patient facing app where a meal reminder is automatically triggered when ketone levels from a continuous ketone monitoring (CKM) device exceed a predefined threshold. In another embodiment, an automatic alert can be sent to the physician or the care team when ketone levels exceed a predefined threshold sustained over time flagging nutritional risk or risk of ketoacidosis. It can also be included in a report for the physician to facilitate a conversation with the patient.

Embodiments of this disclosure can provide visual feedback from CGM-derived metrics to motivate GLP-1 RA adherence. GLP-1 RA improves glucose control by improving fasting glucose and postprandial glucose variability. Progress in glucose control can be included on a dashboard or in personalized reports as improving trends in CGM-derived metrics like TIR, time above 140 mg/dl (TA140), TAR, MAGE, AUC, reduction in hyperglycemic and hypoglycemic episodes. This can motivate continued adherence to the therapy, provide positive reinforcement for dietary and behavioral changes, and help users track progress over time. This can be particularly helpful for GLP-1 RA users facing GI side effects or who are on the therapy for weight management where the effect may not be immediately evident.

Embodiments of this disclosure can monitor alcohol intake GI discomfort is a common side effect of GLP-1 RAs especially during the initial few months which can be worsened by alcohol consumption. Therefore, reducing alcohol consumption can reduce GI discomfort while on GLP-1 RA therapy. Alerts based on continuous alcohol sensor can alert users (e.g., ‘alcohol may cause or worsen nausea while on GLP-1 RA’) and prompt users to take preventative actions before GI symptoms worsen such as sipping water or drinks to help with nausea. Continuous data from alcohol sensors can also be used for positive reinforcement when alcohol is avoided or when alcohol consumption is reduced. For example, alcohol consumption frequency can be monitored continuously over one-week increments and presented to the user as weekly alcohol consumption frequency trends. Offering such insights about the progress can serve as a motivational tool, encouraging further reduction in alcohol intake and encouraging adherence to GLP-1 RA therapy.

Data from alcohol sensor can also be combined with symptoms related to GI discomfort and used to provide insights to the user on avoiding alcohol with certain food types or at particular mealtimes or avoid certain types of alcohol. Finally, if the alcohol consumption rates are high then the system may notify or alert the clinician or the care team for a review and conversation with the patient about addressing alcohol intake and its effect on GI symptoms with GLP-1 RA.

Embodiments of this disclosure can encourage physical activity. GLP-1 RAs are effective for weight loss but reduction in muscle mass is a common concern especially with significant weight loss. Encouraging physical activity, especially anaerobic exercise in GLP-1 RA users can reduce muscle mass loss. Anaerobic exercise stimulates muscle protein synthesis and thus can play a vital role in reducing muscle loss during weight loss therapies such as GLP-1 RA. Lactate levels rise during anaerobic exercise, hence tracking lactate levels during physical activity can be used to monitor if the patient is engaging is sufficient and appropriate physical activity. Lactate levels can also be monitored to optimize training intensity and prevent overtraining. For example, a high lactate level sustained over a longer period can indicate overtraining or insufficient recovery. Such insights can be included in apps and reports for the patients and physicians.

A real-world example will now be presented to help one better understand the disclosed concepts. In one representative deployment, at least some of the disclosed embodiments are implemented within a diabetes and metabolic care clinic that manages patients undergoing GLP-1 receptor agonist therapy. In this setting, patients wear continuous glucose monitors (CGMs) adhered to their upper arm or abdomen, and optionally wear additional analyte sensors (e.g., ketone or lactate monitors). Each patient carries a smartphone that hosts an application corresponding to device 125, while a cloud-hosted clinical service corresponds to service 105. The clinic’s healthcare providers access patient-specific reports and recommendations through a web-based clinical interface connected to the same service infrastructure.

During normal operation, the CGM periodically measures interstitial glucose levels and transmits those measurements via a short-range wireless protocol to the patient’s smartphone. The smartphone application aggregates these measurements into a glucose profile and forwards the data to the cloud service when network connectivity is available. In parallel, the patient may input dietary information into the application (e.g., logging meals or capturing images of food), and the application associates timestamps with both the dietary data and the glucose measurements. The cloud service receives these data streams and stores them as part of a longitudinal patient profile that also includes electronic health record data and medication information previously entered or synchronized. The service then applies the machine learning (ML) model to this combined dataset to generate recommendations, such as suggested insulin dose reductions prior to initiation of GLP-1 RA therapy or adjustments to GLP-1 dosage during treatment.

In one operational instance, a patient begins wearing the CGM for a baseline period prior to starting GLP-1 RA therapy. During this time, the smartphone application may operate in a mode that limits or delays display of glucose readings to the patient, while still transmitting the collected data to the cloud service. The service processes this baseline data to establish a glucose pattern profile. After the baseline period, the application transitions to displaying real-time glucose information, and additional data continues to be transmitted. When the patient initiates GLP-1 RA therapy, the application records a timestamp and dosage information, which is transmitted to the service and tagged within the patient’s glucose profile. As subsequent glucose data is received, the ML model evaluates changes in glycemic patterns relative to the baseline and generates updated recommendations, which are transmitted back to the smartphone application and optionally surfaced to the clinician through a report interface.

The data flow in this deployment is continuous and bidirectional. Sensor data flows from the wearable monitor to the smartphone, and then to the cloud service. Supplemental data, such as dietary inputs or activity logs, are entered through the smartphone and transmitted alongside sensor data. The cloud service processes these inputs using the ML model and generates output data that includes recommendations and insights. These outputs are transmitted back to the smartphone for user display and to the clinical interface for provider review. In some cases, the service may also communicate with an insulin pump associated with the patient to adjust insulin delivery parameters based on the generated recommendation, thereby closing the loop between sensing, analysis, and actuation.

At least some of the disclosed embodiments produce tangible technical behavior within this environment. For example, during the baseline and treatment phases, the smartphone maintains a rolling buffer of recent glucose measurements in local memory while asynchronously transmitting batched data to the cloud service. This batching reduces the frequency of network transmissions, thereby lowering bandwidth utilization and minimizing radio wake cycles on the mobile device. On the cloud side, the ML model operates on aggregated multi-modal data rather than raw continuous streams, which reduces processor overhead associated with handling high-frequency sensor inputs and allows the model to execute on consolidated datasets stored in memory. Additionally, when intermittent CGM data gaps occur, the service generates predicted glucose values that are stored as distinct data representations (e.g., flagged or differentiated entries) within the glucose profile. This reduces the need for continuous sensor sampling while preserving continuity in the dataset, thereby decreasing sensor communication events and associated processing on both the device and service. These coordinated changes in data buffering, transmission frequency, and representation directly affect processor workload, memory residency, and network usage, demonstrating concrete technical operation within a deployed medical monitoring system consistent with practical application under 35 U.S.C. §101.

Attention will now be directed to FIG. 12B, which illustrates a flowchart of an example method 1200 for treating a user by recommending individualized insulin dose adjustments (e.g. reductions) prior to initiation of a therapy involving using a glucagon-like peptide-1 receptor agonist (GLP-1 RA) treatment, where the GLP-1 RA treatment is designed to treat a physiological condition of the user, and where a wearable monitor measures a physiological level of the user. The measured physiological level being used to facilitate the GLP-1 RA treatment. Method 1200 can be implemented by service 105 within architecture 100 of FIG. 1. In other examples, method 1200 can be implemented by device 125 or cloud 115 within architecture 100 of FIG. 1.

In some implementations, the system is specifically configured to generate recommendations prior to initiation of a therapy, such that baseline physiological and behavioral data are used to determine initial therapy parameters. This pre-initiation analysis may reduce the need for reactive adjustments after therapy begins and enables proactive optimization based on predicted responses.

Method 1200 includes an act (act 1205) of using the wearable monitor to measure the physiological level in a body of the user. The physiological level corresponds to a level of at least one of: glucose ketone, lactate, and/or alcohol.

Act 1210 includes optionally accessing dietary data from a food application in which the user entered at least some of the dietary data.

Act 1215 includes accessing a machine learning (ML) model that is trained on multi-modal sources, including one or more of an electronic health record, the physiological level, the dietary data, and/or clinical study datasets that capture insulin dosing patterns before and after GLP-1 RA initiation.

Act 1220 includes providing input to the ML model. The input can include demographic information for the user, a current insulin regimen for the user, a GLP-1 RA type and medication dosage regimen (e.g., insulin information such as insulin type, dose schedule, dose amounts; other medication information, including metformin, Meglitinides (Meg), Sulfonylureas (SU) information) of the user, medical history of the user, and/or diabetes-related laboratory values of the user. The input can also include the dietary data of the user.

Act 1225 includes receiving, from the ML model, output. The output includes a recommendation comprising a recommended a therapy adjustment (e.g., reduction of insulin dosage amounts, removal of Meg or SU). The embodiments can then facilitate treating (act 1230) the user based on the recommended therapy adjustment.

In some implementations, the recommendation need not directly specify a dosage modification. For example, the recommendation may instead indicate whether a therapy should be initiated, delayed, continued, paused, or discontinued, or may provide a comparative evaluation between multiple therapy options. In other scenarios, the recommendation may indicate that insufficient data is available to support a modification, thereby deferring action. Accordingly, the recommendation may represent a decision, a suggestion, a ranking, or an informational output rather than a specific dosage change.

In some implementations, the recommendation may be generated based on multiple objectives, such as minimizing variability, improving time in range, and maintaining user-specific targets. The model may balance these objectives based on learned relationships rather than optimizing a single predefined outcome.

In some implementations, the output of the model may be continuous, discrete, or categorical in nature. For example, the output may include a numerical value, a probability distribution, a classification label, or a set of candidate actions. In some cases, the output may represent an intermediate result that is further processed before being presented as a recommendation.

### Computer systems, computer readable mediums and computer programs

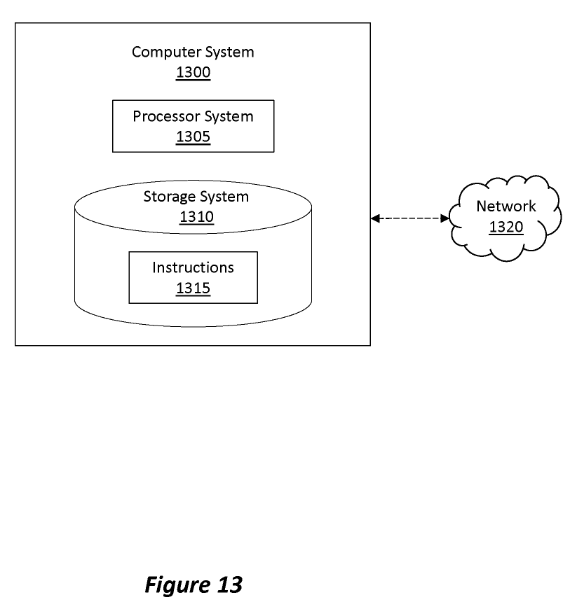

Attention will now be directed to FIG. 13 which illustrates an example computer system 1300 that may include and/or be used to perform any of the operations described herein. For instance, computer system 1300 can implement any of the services described herein, including service 105 of FIG. 1.

Computer system 1300 may take various different forms. For example, computer system 1300 may be embodied as a tablet, a desktop, a laptop, a mobile device, or a standalone device, such as those described throughout this disclosure. Computer system 1300 may also be a distributed system that includes one or more connected computing components/devices that are in communication with computer system 1300. Sensor 130A, device 125 and cloud 115 of FIG. 1 are particular types of computer system 1300.

In its most basic configuration, computer system 1300 includes various different components. FIG. 13 shows that computer system 1300 includes a processor system 1305 that includes one or more processors (aka a “hardware processing unit”) and a storage system 1310.

Regarding the processor(s) of the processor system 1305, it will be appreciated that the functionality described herein can be performed, at least in part, by one or more hardware logic components. For example, and without limitation, illustrative types of hardware logic components/processors that can be used include Field-Programmable Gate Arrays (“FPGA”), Program-Specific or Application-Specific Integrated Circuits (“ASIC”), Program-Specific Standard Products (“ASSP”), System-On-A-Chip Systems (“SOC”), Complex Programmable Logic Devices (“CPLD”), Central Processing Units (“CPU”), Graphical Processing Units (“GPU”), or any other type of programmable hardware.

As used herein, the terms “executable module,” “executable component,” “component,” “module,” “service,” or “engine” can refer to hardware processing units or to software objects, routines, or methods that may be executed on computer system 1300. The different components, modules, engines, and services described herein may be implemented as objects or processors that execute on computer system 1300 (e.g. as separate threads).

Storage system 1310 may be physical system memory, which may be volatile, non-volatile, or some combination of the two. The term “memory” may also be used herein to refer to non-volatile mass storage such as physical storage media. If computer system 1300 is distributed, the processing, memory, and/or storage capability may be distributed as well.

Storage system 1310 is shown as including executable instructions 1315. The executable instructions 1315 represent instructions that are executable by the processor(s) of the processor system 1305 to perform the disclosed operations, such as those described in the various methods.

The disclosed embodiments may comprise or utilize a special-purpose or general-purpose computer including computer hardware, such as, for example, one or more processors and system memory, as discussed in greater detail below. Embodiments also include physical and other computer-readable media for carrying or storing computer-executable instructions and/or data structures. Such computer-readable media can be any available media that can be accessed by a general-purpose or special-purpose computer system. Computer-readable media that store computer-executable instructions in the form of data are “physical computer storage media” or a “hardware storage device.” Furthermore, computer-readable storage media, which includes physical computer storage media and hardware storage devices, exclude signals, carrier waves, and propagating signals. On the other hand, computer-readable media that carry computer-executable instructions are “transmission media” and include signals, carrier waves, and propagating signals. Thus, by way of example and not limitation, the current embodiments can comprise at least two distinctly different kinds of computer-readable media: computer storage media and transmission media.

Computer storage media (aka “hardware storage device”) are computer-readable hardware storage devices, such as RAM, ROM, EEPROM, CD-ROM, solid state drives (“SSD”) that are based on RAM, Flash memory, phase-change memory (“PCM”), or other types of memory, or other optical disk storage, magnetic disk storage or other magnetic storage devices, or any other medium that can be used to store desired program code means in the form of computer-executable instructions, data, or data structures and that can be accessed by a general-purpose or special-purpose computer.

Computer system 1300 may also be connected (via a wired or wireless connection) to external sensors (e.g., one or more remote cameras) or devices via a network 1320. For example, computer system 1300 can communicate with any number of devices or cloud services to obtain or process data. In some cases, network 1320 may itself be a cloud network. Furthermore, computer system 1300 may also be connected through one or more wired or wireless networks to remote/separate computer systems(s) that are configured to perform any of the processing described with regard to computer system 1300.

A “network,” like network 1320, is defined as one or more data links and/or data switches that enable the transport of electronic data between computer systems, modules, and/or other electronic devices. When information is transferred, or provided, over a network (either hardwired, wireless, or a combination of hardwired and wireless) to a computer, the computer properly views the connection as a transmission medium. Computer system 1300 will include one or more communication channels that are used to communicate with the network 1320. Transmissions media include a network that can be used to carry data or desired program code means in the form of computer-executable instructions or in the form of data structures. Further, these computer-executable instructions can be accessed by a general-purpose or special-purpose computer. Combinations of the above should also be included within the scope of computer-readable media.

Upon reaching various computer system components, program code means in the form of computer-executable instructions or data structures can be transferred automatically from transmission media to computer storage media (or vice versa). For example, computer-executable instructions or data structures received over a network or data link can be buffered in RAM within a network interface module (e.g., a network interface card or “NIC”) and then eventually transferred to computer system RAM and/or to less volatile computer storage media at a computer system. Thus, it should be understood that computer storage media can be included in computer system components that also (or even primarily) utilize transmission media.

Computer-executable (or computer-interpretable) instructions comprise, for example, instructions that cause a general-purpose computer, special-purpose computer, or special-purpose processing device to perform a certain function or group of functions. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the embodiments may be practiced in network computing environments with many types of computer system configurations, including personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, and the like. The embodiments may also be practiced in distributed system environments where local and remote computer systems that are linked (either by hardwired data links, wireless data links, or by a combination of hardwired and wireless data links) through a network each perform tasks (e.g. cloud computing, cloud services and the like). In a distributed system environment, program modules may be located in both local and remote memory storage devices.

The present invention may be embodied in other specific forms without departing from its characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

### Glossary

### General features

A “dose” as used herein is a single administration of a pharmaceutical or medicament such as insulin, GLP-1 RA, Meglitinides or Sulfonylureas. An “individualized dose” is a dose specifically tailored for an individual user.

A “dosage”, “dosage level” and “dosage regimen” as used herein describe how doses are regularly administered to a user as part of an on-going therapy. Dosages may include the type of pharmaceutical or medicament, the dose schedule and the dose amount.

To “titrate” as used herein means to adjust a dosage over time. In particular “titrate up” means to increase the dosage over time, while “titrate down” means to decrease the dosage over time. The relevant time period of titration (i.e. the time over which the dosage is adjusted) is referred to as the titration period.

The term “multi modal” as used herein indicates that data has been obtained from a plurality of different sources (i.e. “multi-modal sources”) and/or of a plurality of different data types.

The terms “machine learning model”, “ML model” and just “ML” as used herein refer to a model where at least a portion thereof has been trained using machine learning. The ML models discussed herein may include one or more sub-models that each provide a different function. In general, where it is stated that the ML model performs more than one different function, it should be assumed that a plurality of sub-models are used. In some instances, the sub-models may themselves be ML models. However, not all functionality provided by the ML model discussed herein may necessarily involve ML, in which case a portion of the sub-models may not be ML. For example, a portion of the sub-models may be based on predetermined rules.

The terms “generative artificial intelligence”, “generative AI” and “gen AI” are a special type of ML model where at least a portion of the output is generative. Examples of generative AI include large language models (“LLMs”).

The term “electronic health record” (“EHR”) is a record of a user’s health. Data contained in an EHR may include information about the user, including demographic information (for example, gender, age, ethnicity), diagnoses (for example, type 2 diabetes, obesity), body measurements (for example, weight, glucose level, other analyte level), any pharmaceuticals or medicaments that the user is actively taking and dosages. Medical history may also be included in the EHR. “Medical history” may include diagnoses, measurements and any pharmaceuticals or medicaments that the user is actively taking and dosages over time.

### GLP-1 features

A “GLP-1 receptor agonist” (GLP-1 RA) is a type of pharmaceutical that mimics natural GLP-1. Natural GLP-1 (Glucagon-like peptide-1) is a type of natural hormone in a human’s body that helps regulate a person’s blood sugar, digestion, and appetite. As used herein, “GLP-1 RA” and “GLP-1” refer to the pharmaceutical, whilst the phrase “natural GLP-1” refers to the naturally occurring human hormone.

“GLP-1 RA treatment” as used herein is a treatment involving doses of GLP-1.

A “GLP-1 RA type” or “GLP-1 type medication” is a reference to the type of GLP-1 pharmaceutical. Type of GLP-1 pharmaceutical may vary by brand. Type of GLP-1 pharmaceutical may be compounded or non-compounded. Type of GLP-1 for the purpose of the present disclosure may also include dual GLP-1 and a gastric inhibitory polypeptide (GIP) treatments.

### Glucose related features

A “glucose level”, also commonly referred to as a “blood sugar” level, is an instantaneous measurement of how much glucose is in a user’s blood, typically in mg/dL. Below certain thresholds, such as 70 mg/dL and 54 mg/DL, complications such as loss of consciousness, cardiovascular disease, retinopathy, neuropathy, and nephropathy can occur. Glucose level is generally measured using a continuous glucose monitor (CGM), also referred to herein as a “sensor” (e.g. sensor 130A) or “sensor control device”. Glucose level data from the CGM is also referred to herein as “sensor data”, “analyte data”, “CGM sensor data”, and “CGM data”.

A “glucose profile”, “glucose trace” and “glucose chart” as used herein is a data series or chart reflective of a user’s glucose levels over a period of time. To determine a glucose profile, glucose levels are measured at set intervals (e.g. one minute, two minutes, three minutes, etc.). Typically the horizontal axis reflects the glucose levels over a minutes-based time period. For example, FIG. 3 shows the horizontal axis going up to 18,000 minutes. Various glucose profiles are shown in FIGS. 3 to 5B , for example. Glucose levels between the set intervals may be estimated using techniques known in the art, meaning that glucose profiles are generally considered “continuous”. The term “intermittent” is used herein with respect to glucose profiles when one or more of the measurements at set intervals has been missed.

A “glycemic pattern” or “glucose pattern” is a pattern in a user’s glucose profile. Glycemic patterns generally reflect the user’s typical glucose level at certain times of day. For example, FIG. 6B shows a user’s average glucose pattern throughout the day over 14 days, The glycemic pattern may occur due to an event such as food intake, physical activity, or administration of an insulin dose. Events may be logged by the user in an application so that the event can be associated with the glycemic pattern.

A “glucose pattern insights report” or “GPI report” indicates patterns of glucose behavior for a user. An example report is shown in FIG. 6B. A GPI report may be relevant to a particular date or time range, which may be indicated on the report. For example, in FIG. 6B the report is for a time range of 14 days, and the date range is Jan 25 to Feb 7 2024. A GPI report may contain glycemic patterns, time in certain glucose ranges (i.e. above or below certain thresholds), and/or glucose statistics. Glucose statistics may include average glucose and a glucose management indicator or GMI.

A “glucose management indicator” or “GMI” is a measure of how well the user is managing their glucose. GMI is calculated from the average glucose value (measured in mg/dL) derived from at least 14 days of continuous glucose monitor (CGM) data. The formula is: GMI (%) = 3.31 + 0.02392 mean glucose (mg/dL). It provides an estimate for A1C, though it is not identical to it.

An “A1C” or “hemoglobin A1C” or “HbA1c” value is a blood test result that represents a user’s average glucose levels over the past 2 to 3 months. The A1C test measures the amount of blood sugar attached to hemoglobin. “Diabetes-related lab values of the user” may include A1C values.

### Ketone, lactate or alcohol related features

A “ketone level” measures measure the amount of fat-produced energy compounds in the blood or urine, with normal levels typically below 0.6 mmol/L. Levels between 0.6–1.5 mmol/L are typically considered slightly high, 1.6–3.0 mmol/L usually indicate risks of Diabetic Ketoacidosis, and over 3.0 mmol/L are considered to signify dangerous, emergency-level, high ketones.

A “lactate level” measures lactic acid in the blood, typically serving as an indicator of whether body tissues are receiving enough oxygen. Normal levels are typically below 2 mmol/L (often < 1 mmol/L), while elevated levels (hyperlactatemia) indicate tissue hypoxia, shock, sepsis, or metabolic stress.

An “alcohol level” measures the amount of alcohol in blood, typically expressed as as a percentage or in milligrams per 100 milliliters of blood. For instance, a blood alcohol level of 0.08% means a person has 0.08 grams of alcohol per 100 milliliters of blood.

### Food related features

The term “dietary data” as used herein includes any information related to the user’s eating habits. Dietary data may include food portions, macronutrient composition (carbohydrates, proteins, fats), and meal timing. Information about portion sizes, macronutrient composition and meal timing in conjunction with the CGM data can be used to assess how specific foods, macronutrients ratios, or meal patterns (e.g., order of food intake, meal frequency, meal times) influence glucose excursions. At least a portion of the dietary data may be entered by the user, for example the user may input images of their food choices (see image data 610 in FIG. 6A and/or image 920 of FIG. 9 for further information). Dietary data may also include missed meals.

In some implementations, the ML model may learn causal or semi-causal relationships between user behaviors (e.g., food choices, meal timing, activity patterns) and physiological responses. For example, the model may identify that a particular sequence of dietary inputs consistently produces a specific analyte response pattern for a given user. These learned relationships may be used to generate recommendations that modify behavior rather than only adjusting therapy.

Dietary data may be obtained through a variety of mechanisms in addition to manual entry. For example, the system may receive dietary information from third-party applications, wearable devices, or image-based analysis of captured food images. In some scenarios, dietary data may be partially inferred based on patterns in analyte responses, location data, or previously logged meals. These different sources of dietary data may be combined or weighted differently depending on their reliability and availability.

The terms “food application” and “food app” as used herein is a type of software application with functionality for allowing the user to input dietary data. For example, the food application may provide an option to upload an image to allow the user to input images of their food choices. App 700, app 800 and/or app 900 may be the food application. FIGS. 7, 8, 9 and 10 illustrate various example user interfaces of the food application. The food application may include other application functionalities, such as the continuous glucose monitoring application functionalities discussed herein and/or service 105.

### System and device features

A “computer system” as used herein is any system with at least one or more processors and one or more hardware storage devices that store instructions that are executable by the one or more processors to cause the computer system to perform the various methods discussed herein. Further features of computer system 1300 are described with respect to FIG. 13. Sensor 130A, device 125 and cloud 115 are particular examples of computer systems that may be used alone or in combination for implementing the methods discussed herein. With reference to FIG. 1, architecture 100 is shown as including various hardware features, such as sensor 130A, applicator 130B, device 125, cloud 115, and insulin pump 145. Other features shown in architecture 100 may be implemented in software, including sensor data 135, service 105, ML engine 110, analytics 120, GPI report 135, and output data 140 (including profile data 140A, predictions 140B, and recommendations 140C). The software features may be implemented on one or more computer systems, for example computer system 1300 discussed with respect to FIG. 13. Sensor 130A, device 125 and cloud 115 are particular examples of computer systems that may be used alone or in combination for implementing the software features as further discussed herein.

A “service” as used herein refers to an automated program (e.g. a computer program) that is tasked with performing different actions based on input. The applications, including specifically the food application, may be implemented as part of the service and/or may be provided by the service. The service may operate on one or more computers (for example, computer system 300, sensor 130A, device 125 and cloud 115).

A “user interface” as discussed herein is generally a graphical user interface to allow interaction between an application (including the food application) and/or service and the user. A “user interface element” as used herein is a graphical element on the graphical user interface to allow for user interaction. For some user interface elements, interaction may be passive, for example the element may display information to the user to assist in managing their glucose level or other analyte level (for example, predicted levels 725, alarm 710, message 715; predicted levels 820, alarm 810, message 815; meal storing 905, meal score 910, glucose level 915). Other user interface elements may enable user interaction.

A “wearable monitor” or “sensor” as used herein is a wearable device capable of measuring an analyte level. Various wearable monitors are discussed herein, including a continuous glucose monitor and monitors of other analyte levels (particularly glucose, lactate and/or alcohol). Wearable monitors may have a limited wear time, referred to herein as a “sensor wear”.

### Numbered embodiments

### Insulin dose aspect

1. A method for recommending an individualized insulin dose for a therapy involving using a glucagon-like peptide-1 receptor agonist (GLP-1 RA) treatment, where the GLP-1 RA treatment is designed to treat a physiological condition of a user, and where a wearable monitor measures a physiological level of the user, the measured physiological level being used to facilitate said GLP-1 RA treatment, said method comprising:
obtaining from the wearable monitor the measured physiological level of a body of the user, wherein the physiological level corresponds to a level of at least one of:
   0glucose,
   ketone,
   lactate, or
   alcohol;
accessing a machine learning (ML) model that is trained on multi-modal sources, the multi-modal sources including one or more of: an electronic health record, and clinical study datasets that capture insulin dosing patterns before and after GLP-1 RA initiation;
providing input to the ML model, wherein the input includes one or more of:
   demographic information for the user,
   a current insulin regimen for the user,
   a GLP-1 RA type and dosage of the user,
   medical history of the user, and
   diabetes-related laboratory values of the user;
receiving, from the ML model, output, wherein the output includes a recommendation comprising a recommended insulin dose adjustment.

2. The method of embodiment 1, wherein the recommended insulin dose adjustment is a recommended insulin dosage reduction.

3. The method of embodiment 1 or 2, further comprising: accessing dietary data from a food application.

4. The method of embodiment 3, wherein the user entered at least some (i.e. a portion) of the dietary data.

5. The method of embodiment 3 or 4, wherein the multi-modal sources further comprise the dietary data.

6. The method of any of embodiments 3-5, wherein the input further comprises dietary data.

7. The method of any preceding embodiment, wherein the multi-modal sources further comprise physiological level.

8. The method of any preceding embodiment, wherein the multi-modal sources further comprise continuous glucose monitoring metrics.

9. The method of any preceding embodiment, wherein the multi-modal sources include electronic health records.

10. The method of any preceding embodiment, wherein the ML model is trained on one or more of: patient demographics, insulin regimen, GLP-1 RA type and dosage, medical history, and diabetes-related laboratory values.

11. The method of embodiment 10, wherein the ML model is trained on patient demographics, insulin regimen, GLP-1 RA type and dosage, medical history, and diabetes-related laboratory values.

12. The method of any preceding embodiment, wherein the method is performed prior to initiation of the therapy involving using the GLP-1 RA treatment.

13. The method of any preceding embodiment, wherein the medical history includes the physiological level.

14. The method of any preceding embodiment, wherein the recommendation further indicates that the GLP-1 RA dosage is to be an increased titration of the GLP-1 RA treatment.

15. The method of any preceding embodiment, wherein the recommendation further indicates an adjustment to an insulin dosage.

16. The method of any preceding embodiment, wherein the recommendation further indicates that the GLP-1 RA dosage is to be a decreased titration of the GLP-1 RA treatment.

17. The method of any preceding embodiment, wherein the ML model further relies on a glucose pattern insights (GPI) report, a glucose management indicator (GMI), and/or A1C value to generate the recommendation.

18. The method of any preceding embodiment, wherein multi-modal sources further comprise one or more of: glucose pattern insights (GPI) report, a glucose management indicator (GMI), and A1C value.

19. The method of any preceding embodiment, further comprising displaying a user option to indicate whether the user has type 1 diabetes, has type 2 diabetes, is on an oral medication, or is on insulin therapy.

20. The method of any preceding embodiment, wherein the ML model is trained to identify one or more of the following detected conditions: a first condition in which an A1C response of the user lowers but a weight of the user does not lower; a second condition in which the weight of the user does lower but the A1C response does not lower; or the user is one who does not respond to GLP-1 type medications.

21. The method of any preceding embodiment, wherein the ML model uses the physiological level measured by the wearable monitor to predict whether the user is one who will respond earlier than a 4-week titration period.

22. The method of embodiment 21, wherein the prediction of the ML model is based on a detected lack of a glycemic response from the user while the user is being treated with the GLP-1 RA treatment.

23. The method of any preceding embodiment, wherein the wearable monitor is a continuous glucose monitor (CGM).

24. The method of any preceding embodiment, further comprising monitoring a gastrointestinal tolerance for the user while the user is being treated with the GLP-1 RA treatment.

25. The method of any preceding embodiment, wherein the wearable monitor is one of a plurality of wearable monitors that are worn by the user.

26. The method of any preceding embodiment, wherein the wearable monitor is worn by the user for at least 14 days prior to the user being treated with the GLP-1 RA treatment for a first time.

27. The method of any preceding embodiment, wherein the wearable monitor is worn by the user for at least 28 days prior to the user being treated with the GLP-1 RA treatment for a first time.

28. The method of any preceding embodiment, wherein the wearable monitor is one of a plurality of wearable monitors that are worn by the user for at least 28 days prior to the user being treated with the GLP-1 RA treatment for a first time.

29. The method of any preceding embodiments, further comprising displaying one or more user interface (UI) elements for entering eating habits of the user.

30. The method of embodiment 29, wherein the eating habits form at least a portion of the dietary data.

31. The method of any preceding embodiment, wherein the ML model is further trained to detect glycemic patterns of the user based on an identified order of food type intake of the user.

32. The method of embodiment 31, wherein the order of food type intake includes an order in which a fatty food is consumed by the user first.

33. The method of embodiment 31 or 32, wherein the order of food type intake includes an order in which a food having at least a threshold level of fiber is consumed by the user first.

34. The method of embodiment 31, 32 or 33, wherein the order of food type intake includes an order in which a food having at least a threshold level of protein is consumed by the user first.

36. The method of any preceding embodiment, wherein the physiological level is the glucose level.

37. The method of any preceding embodiment, wherein the ML model is further trained to predict whether the user has prediabetes.

38. The method of any preceding embodiment, wherein the ML model detects that an appetite of the user is likely reduced subsequent to treatment using the GLP-1 RA treatment based on a detected change to a glycemic pattern, which is reflected in a glucose profile.

39. The method of any preceding embodiment, further comprising modifying an insulin dispersal rate of an insulin pump of the user based on a glucose profile and based on the recommendation generated by the ML model.

40. A computer system comprising: one or more processors; and one or more hardware storage devices that store instructions that are executable by the one or more processors to cause the computer system to perform the method of any preceding embodiment.

41. The method of embodiment 40, wherein the computer system is implemented in a device and/or a cloud.

42. The method of embodiment 40 or 41, wherein the food application is installed on the computer system.

43. A system comprising: the computer system of embodiment 40; and a wearable monitor configured to measure a physiological level of the user.

44. A computer program comprising instructions which, when executed by one or more processors, cause the one or more processors to perform the method of any one of embodiments 1 to 39.

45. A computer readable medium comprising instructions which, when executed by one or more processors, cause the one or more processors to perform the method of any one of embodiments 1 to 39.

### GLP-1 RA dose aspect

46. A method for using a machine learning (ML) model to recommend continuance or modification of a usage of a glucagon-like peptide-1 (GLP-1) treatment of a user, where the GLP-1 treatment is designed to treat a physiological condition of the user, and where one or more continuous glucose monitors (CGMs) measure glucose levels of the user, the measured glucose levels being used to facilitate the ML model's recommendation regarding continuance or modification of the GLP-1 treatment, said method including:
operating in a blinded mode during a first period of time in which the one or more CGMs measure the glucose levels in a body of the user, wherein operating in the blinded mode prevents display of measurements obtained by the one or more CGMs during the first period of time, and wherein the measured glucose levels obtained during the first period of time are included in a glucose profile for the user;
subsequent to the first period of time, operating in a non-blinded mode during a second period of time in which the one or more CGMs measure the glucose levels in the user's body, wherein operating in the non-blinded mode enables display of measurements obtained by the one or more CGMs during the second period of time, and wherein the measured glucose levels obtained during the second period of time are included in the glucose profile for the user;
determining that the user has initiated the GLP-1 treatment, wherein the GLP-1 treatment involves a first dosage level of a GLP-1;
adding tagged data to the glucose profile for the user, wherein the tagged data includes a timestamp reflective of when the user initiated the GLP-1 treatment and includes data reflective of the first dosage level;
during a third period of time subsequent to the second period of time, using the one or more CGMs to measure the glucose levels of the user's body while the user is undergoing the GLP-1 treatment, wherein the measured glucose levels obtained during the third period of time are included in the glucose profile for the user; and
in response to the ML model detecting a change to a glycemic pattern of the user during the third period of time (e.g., the user can continue to wear the monitor for any number of time periods, without limit, to obtain insights regarding lifestyle, food, etc.), where the change to the glycemic pattern is reflected in the glucose profile, causing the ML model to generate a recommendation for the user to have a particular dosage level of the GLP-1 based on the detected change to the glycemic pattern.

47. The method of embodiment 46, wherein the recommendation indicates that the particular dosage level of the GLP-1 is to be an increased titration of the GLP-1.

48. The method of embodiment 46, wherein the recommendation indicates that the particular dosage level of the GLP-1 is to be a decreased titration of the GLP-1 or, alternatively, an adjustment to an insulin level (e.g., a decreased titration of insulin).

49. The method of any of embodiments 46 to 48, wherein the ML model further relies on a glucose pattern insights (GPI) report to generate the recommendation.

50. The method of any of embodiments 46 to 49, further comprising displaying a user option to indicate whether the user has type 1 diabetes, has type 2 diabetes, is on an oral medication, or is on basal insulin.

51. The method of any of embodiments 46 to 50, wherein the ML model is trained to identify one or more of the following detected conditions: a first condition in which an A1C response of the user lowers but a weight of the user does not lower; a second condition in which the weight of the user does lower but the A1C response does not lower; or the user is one who does not respond to GLP-1 type medications.

52. The method of any of embodiments 46 to 51, wherein the ML model uses the glucose levels measured by the one or more CGMs to predict whether the user is one who will respond earlier than a 4 week titration period. In some implementations, the system does not rely on estimating or modeling intermediary physiological processes such as gastric emptying rates. Instead, the ML model operates directly on measured and derived features to generate recommendations. This allows the system to avoid dependency on inferred physiological proxies and to instead utilize observed data and learned relationships.

53. The method of embodiment 52, wherein the prediction of the ML model is based on a detected lack of a glycemic response from the user while the user is being treated with the GLP-1.

54. The method of any of embodiments 46 to 51, wherein the first period of time is a minimum of 14 days.

55. The method of embodiment 54, further comprising, after the first period of time finishes, triggering a questionnaire for the user to complete.

56. The method of embodiment 54 or 55, wherein the first period of time is at least 28 days.

57. The method of any of embodiments 46 to 51, wherein the blinded mode has a duration of one sensor wear.

58. The method of any of embodiment 46 to 51, wherein the blinded mode has a duration of more than one sensor wear.

59. The method of embodiment 58, wherein the blinded mode has a duration of two sensor wears.

60. The method of embodiment 57, 58 or 59, wherein the sensor wear duration is 14 days.

61. The method of any of embodiments 46 to 60, further comprising monitoring a gastrointestinal tolerance for the user while the user is being treated with the GLP-1.

62. The method of any of embodiments 46 to 61, wherein the one or more CGMs are worn by the user for at least 28 day prior to the user being treated with the GLP-1 for a first time.

63. The method of any of embodiments 46 to 62, wherein the method includes displaying one or more user interface (UI) elements for entering eating habits of the user by tagging the glucose profile with supplement data reflective of those eating habits.

64. The method of any of embodiments 46 to 63, wherein the ML model is further trained to detect glycemic patterns of the user based on an identified order of food type intake of the user.

65. The method of embodiment 64, wherein the order of food type intake includes an order in which a fatty food is consumed by the user first.

66. The method of embodiment 64 or 65, wherein the order of food type intake includes an order in which a food having at least a threshold level of fiber is consumer by the user first.

67. The method of embodiment 64, 65 or 66, wherein the order of food type intake includes an order in which a food having at least a threshold level of protein is consumer by the user first.

68. The method of any of embodiments 46 to 67, wherein 2 weeks of blinded data and 2 weeks of non-blinded data are obtained.

69. The method of embodiment 68, wherein initiation of the GLP-1 treatment starts after the 2 weeks of blinded data and the 2 weeks of non-blinded data are obtained.

70. The method of any of embodiments 46 to 69, wherein the ML model is further trained to predict whether the user has prediabetes based on the glucose profile.

71. The method of any of embodiments 46 to 70, wherein the ML model detects that an appetite of the user is likely reduced subsequent to treatment using the GLP-1 based on the detected change to the glycemic pattern.

72. The method of embodiment 71, wherein the detected change to the glycemic pattern is reflected in the glucose profile.

73. The method of any of embodiments 46 to 72, further including modifying an insulin dispersal rate of an insulin pump of the user based on the glucose profile and based on the recommendation generated by the ML model.

74. The method of any of embodiments 46 to 73, wherein insulin doses of a patient on long-acting insulin only or multiple daily injections is modified based on the glucose profile and based on the recommendation generated by the ML model.

75. The method of any of embodiments 46 to 74, wherein, after undergoing the GLP-1 treatment, the ML model detects a new pattern of insulin injection provided by an insulin pump used by the user.

76. The method of any of embodiments 46 to 75, further comprising adjusting an insulin dose for MDI and/or basal-insulin users.

77. The method of any of embodiments 46 to 76, wherein the ML model is a supervised ML model, and wherein patterns and weight loss are tagged in the glucose profile and used by the supervised ML model.

78. The method of any of embodiments 46 to 77, further comprising tagging and recording a second dosing of the GLP-1 in the glucose profile.

79. The method of any of embodiments 46 to 78, further including generating a glucose pattern insights (GPI) report including patterns of glucose behavior for the user, wherein the ML model determines a treatment protocol based on the patterns in the GPI report.

80. The method of any of embodiments 46 to 79, further comprising displaying a user interface element to enter mealtimes of the user.

81. The method of any of embodiments 46 to 80, wherein the ML model predicts that the user should titrate up in dosing the GLP-1, and wherein the method includes displaying a predicted titration rate generated by the ML model.

82. The method of any of embodiments 46 to 81, wherein the ML model predicts that the user should titrate down in dosing the GLP-1, and wherein the method includes displaying a predicted titration rate generated by the ML model.

83. The method of any of embodiments 46 to 82, further comprising displaying a first option for the user to enter a workout performed by the user and includes displaying a second option to tag the user's glucose profile based on a timing of the workout.

84. The method of any of embodiments 46 to 83, wherein the ML model includes generative artificial intelligence (AI), wherein an image of food consumed by the user is obtained, and wherein the generative AI determines a nutrient value of the food based on the image.

85. The method of any of embodiments 46 to 84, wherein the ML model includes generative artificial intelligence (AI), wherein an image of food consumed by the user is obtained, and wherein the generative AI determines a food pattern of the user.

86. The method of any of embodiments 46 to 84, wherein the ML model includes generative artificial intelligence (AI), wherein an image of food consumed by the user is obtained, and wherein the generative AI determines whether a food pattern of the user has changed over time while the user is undergoing the GLP-1 treatment.

87. The method of any of embodiments 46 to 86, further comprising displaying an option to track a nausea level of the user while the user is undergoing the GLP-1 treatment.

88. The method of any of embodiments 46 to 87, wherein the method includes displaying an option to track one or more of a sleep rate, a heartrate, a medication consumption rate, or a bowel movement rate of the user while the user is undergoing the GLP-1 treatment.

89. The method of any of embodiments 46 to 88, further comprising communicating with a wearable device worn by the user, the wearable device detecting activities of the user, wherein the method includes receiving data reflective of the activities, and wherein the method includes tagging the glucose profile with the data reflective of the activities.

90. The method of any of embodiments 46 to 89, wherein the recommendation further includes a recommendation to switch from undergoing the GLP-1 treatment to undergoing a dual GLP-1 and a gastric inhibitory polypeptide (GIP) drug treatment.

91. The method of any of embodiments 46 to 89, wherein the recommendation is to switch from one GLP-1 RA to another one (e.g., from one brand/type to another brand/type, from a compounded GLP-1 to a non-compounded GLP-1, from a non-compounded GLP-1 to a compounded GLP-1).

92. The method of any of embodiments 46 to 91, wherein the one or more CGMs are used intermittently at least during one of the first period of time or the second period of time.

93. The method of any of embodiment 92, wherein the ML model is further configured to predict glucose levels during the intermittent use of the one or more CGMs.

94. The method of embodiment 93, wherein the ML model generates the recommendation based, at least in part, on the predicted glucose levels.

In some implementations, recommendations may be generated based on predicted future physiological states rather than solely on observed measurements. For example, the model may forecast future analyte trajectories based on current and historical inputs and generate recommendations intended to influence those predicted trajectories.

95. The method of any of embodiments 46 to 94, further comprising: determining that the one or more CGMs were used intermittently at least during one of the first or second periods of time; causing the ML model to generate predicted glucose levels during time periods when the one or more CGMs were used intermittently,, wherein determining that the user has initiated the GLP-1 treatment is subsequent to the second period of time, wherein causing the ML model to generate a recommendation for the user to have a particular dosage level of the GLP-1 is further based on the predicted glucose levels.

96. A method for using a machine learning (ML) model to recommend continuance or modification of a usage of a glucagon-like peptide-1 (GLP-1) treatment of a user, where the GLP-1 treatment is designed to treat a physiological condition of the user, and where one or more continuous glucose monitors (CGMs) measure glucose levels of the user, the measured glucose levels being used to facilitate the ML model's recommendation regarding continuance or modification of the GLP-1 treatment, said method being implemented by a service and including: operating in a blinded mode during a first period of time in which the one or more CGMs measure the glucose levels in a body of the user, wherein operating in the blinded mode prevents display of measurements obtained by the one or more CGMs during the first period of time, and wherein the measured glucose levels obtained during the first period of time are included in a glucose profile for the user; subsequent to the first period of time, operating in a non-blinded mode during a second period of time in which the one or more CGMs measure the glucose levels in the user's body, wherein operating in the non-blinded mode enables display of measurements obtained by the one or more CGMs during the second period of time, and wherein the measured glucose levels obtained during the second period of time are included in the glucose profile for the user; subsequent to the second period of time, determining that the user has initiated the GLP-1 treatment, wherein the GLP-1 treatment involves a first dosage level of a GLP-1; adding tagged data to the glucose profile for the user, wherein the tagged data includes a timestamp reflective of when the user initiated the GLP-1 treatment and includes data reflective of the first dosage level; during a third period of time subsequent to the second period of time, using the one or more CGMs to measure the glucose levels of the user's body while the user is undergoing the GLP-1 treatment, wherein the measured glucose levels obtained during the third period of time are included in the glucose profile for the user; in response to the ML model detecting a change to a glycemic pattern of the user during the third period of time, where the change to the glycemic pattern is reflected in the glucose profile, causing the ML model to generate a recommendation for the user to either titrate up or titrate down the GLP-1 based on the detected change to the glycemic pattern, wherein the recommendation includes a specific titration amount for the user.

97. A method for using a machine learning (ML) model to recommend continuance or modification of a usage of a glucagon-like peptide-1 (GLP-1) treatment of a user, where the GLP-1 treatment is designed to treat a physiological condition of the user, and where one or more continuous glucose monitors (CGMs) measure glucose levels of the user, the measured glucose levels being used to facilitate the ML model's recommendation regarding continuance or modification of the GLP-1 treatment, said method including: determining that the user has initiated the GLP-1 treatment, wherein the GLP-1 treatment involves a first dosage level of a GLP-1; adding tagged data to a glucose profile for the user, wherein the tagged data includes a timestamp reflective of when the user initiated the GLP-1 treatment and includes data reflective of the first dosage level; using the one or more CGMs to measure glucose levels of a body of the user while the user is undergoing the GLP-1 treatment, wherein the measured glucose levels are included in the glucose profile for the user; and in response to the ML model detecting, within the glucose profile, a glycemic pattern of the user, causing the ML model to generate a recommendation for the user to have a particular dosage level of the GLP-1 based on the detected glycemic pattern.

98. A system for using a machine learning (ML) model to recommend continuance or modification of a usage of a glucagon-like peptide-1 (GLP-1) treatment of a user, where the GLP-1 treatment is designed to treat a physiological condition of the user, the system including: one or more continuous glucose monitors (CGMs) configured to measure glucose levels of the user, the monitored measured glucose levels being used to facilitate the ML model's recommendation regarding continuance or modification of the GLP-1 treatment; and one or more processors configured to cause the system perform the method of any of embodiments 46 to 97.

99. A computer-readable medium for using a machine learning (ML) model to recommend continuance or modification of a usage of a glucagon-like peptide-1 (GLP-1) treatment of a user, where the GLP-1 treatment is designed to treat a physiological condition of the user, the computer-readable medium comprising instructions which, when executed by the processor, cause the processor to perform the method of any of embodiments 46 to 97 in conjunction with one or more glucose monitors (GCMs), wherein the one or more GCMs are configured to measure glucose levels of the user, the monitored measured glucose levels being used to facilitate the ML model’s recommendation regarding continuance or modification of the GLP-1 treatment.

100. A computer program for using a machine learning (ML) model to recommend continuance or modification of a usage of a glucagon-like peptide-1 (GLP-1) treatment of a user, where the GLP-1 treatment is designed to treat a physiological condition of the user, the computer program comprising instructions which, when executed by the processor, cause the processor to perform the method of any of embodiments 46 to 97 in conjunction with one or more glucose monitors (GCMs), wherein the one or more GCMs are configured to measure glucose levels of the user, the monitored measured glucose levels being used to facilitate the ML model’s recommendation regarding continuance or modification of the GLP-1 treatment.

101. A computer-readable medium for using a machine learning (ML) model to recommend continuance or modification in conjunction with one or more glucose monitors (GCMs), wherein the one or more GCMs are configured to measure glucose levels of the user, the monitored measured glucose levels being used to facilitate the ML model's recommendation regarding continuance or modification of the GLP-1 treatment.

102. A computer program for using a machine learning (ML) model to recommend continuance or modification in conjunction with one or more glucose monitors (GCMs), wherein the one or more GCMs are configured to measure glucose levels of the user, the monitored measured glucose levels being used to facilitate the ML model's recommendation regarding continuance or modification of the GLP-1 treatment.

What is claimed is:

1. A method for treating a user by recommending individualized insulin dose reductions prior to initiation of a therapy involving using a glucagon-like peptide-1 receptor agonist (GLP-1 RA) treatment, where the GLP-1 RA treatment is designed to treat a physiological condition of the user, and where a wearable monitor measures a physiological level of the user, the measured physiological level being used to facilitate said GLP-1 RA treatment, said method comprising:
using the wearable monitor to measure the physiological level in a body of the user, wherein the physiological level corresponds to a level of at least one of: glucose, ketone, lactate, or alcohol;
accessing dietary data from a food application in which the user entered at least some of the dietary data;
accessing a machine learning (ML) model that is trained on multi-modal sources, including an electronic health record, the physiological level, the dietary data, and clinical study datasets that capture insulin dosing patterns before and after GLP-1 RA initiation;
providing input to the ML model, wherein the input includes demographic information for the user, a current insulin regimen for the user, a GLP-1 RA type and dosage of the user, medical history of the user, the dietary data, and diabetes-related laboratory values of the user;
receiving, from the ML model, output, wherein the output includes a recommendation comprising a recommended insulin dose adjustment.

2. The method of claim 1, wherein the recommendation indicates that the GLP-1 RA dosage is to be an increased titration of the GLP-1 RA treatment.

3. The method of claim 1, wherein the recommendation indicates that the GLP-1 RA dosage is to be a decreased titration of the GLP-1.

4. The method of claim 1, wherein the ML model further relies on a glucose pattern insights (GPI) report, a glucose management indicator (GMI), or A1C value to generate the recommendation.

5. The method of claim 1, wherein the method includes displaying a user option to indicate whether the user has type 1 diabetes, has type 2 diabetes, is on an oral medication, or is on insulin therapy.

6. The method of claim 1, wherein the ML model is trained to identify one or more of the following detected conditions: a first condition in which an A1C response of the user lowers but a weight of the user does not lower; a second condition in which the weight of the user does lower but the A1C response does not lower; or the user is one who does not respond to GLP-1 type medications.

7. The method of claim 1, wherein the ML model uses the physiological level measured by the wearable monitor to predict whether the user is one who will respond earlier than a 4-week titration period, and wherein the prediction of the ML model is based on a detected lack of a glycemic response from the user while the user is being treated with the GLP-1 RA treatment.

8. The method of claim 1, wherein the wearable monitor is a continuous glucose monitor (CGM).

9. The method of claim 1, wherein the method includes monitoring a gastrointestinal tolerance for the user while the user is being treated with the GLP-1 RA treatment.

10. The method of claim 1, wherein the wearable monitor is one of a plurality of wearable monitors that are worn by the user for at least 28 days prior to the user being treated with the GLP-1 RA treatment for a first time.

11. The method of claim 1, wherein the method includes displaying one or more user interface (UI) elements for entering eating habits of the user.

12. The method of claim 11, wherein the ML model is further trained to detect glycemic patterns of the user based on an identified order of food type intake of the user.

13. The method of claim 12, wherein the order of food type intake includes an order in which a fatty food is consumed by the user first.

14. The method of claim 12, wherein the order of food type intake includes an order in which a food having at least a threshold level of fiber is consumed by the user first.

15. The method of claim 12, wherein the order of food type intake includes an order in which a food having at least a threshold level of protein is consumed by the user first.

16. The method of claim 1, wherein the physiological level is the glucose.

17. The method of claim 1, wherein the ML model is further trained to predict whether the user has prediabetes.

18. The method of claim 1, wherein the ML model detects that an appetite of the user is likely reduced subsequent to treatment using the GLP-1 RA treatment based on a detected change to a glycemic pattern, which is reflected in a glucose profile.

19. The method of claim 1, further comprising modifying an insulin dispersal rate of an insulin pump of the user based on a glucose profile and based on the recommendation generated by the ML model.

20. A computer system that treats a user by recommending individualized insulin dose reductions prior to initiation of a therapy involving using a glucagon-like peptide-1 receptor agonist (GLP-1 RA) treatment, where the GLP-1 RA treatment is designed to treat a physiological condition of the user, and where a wearable monitor measures a physiological level of the user, the measured physiological level being used to facilitate said GLP-1 RA treatment, said computer system comprising:
one or more processors; and
one or more hardware storage devices that store instructions that are executable by the one or more processors to cause the computer system to:
use the wearable monitor to measure the physiological level in a body of the user, wherein the physiological level corresponds to a level of at least one of: glucose, ketone, lactate, or alcohol;
access dietary data from a food application in which the user entered at least some of the dietary data;
access a machine learning (ML) model that is trained on multi-modal sources, including an electronic health record, the physiological level, the dietary data, and clinical study datasets that capture insulin dosing patterns before and after GLP-1 RA initiation;
provide input to the ML model, wherein the input includes demographic information for the user, a current insulin regimen for the user, a GLP-1 RA type and dosage of the user, medical history of the user, the dietary data, and diabetes-related laboratory values of the user;
receive, from the ML model, output, wherein the output includes a recommendation comprising a recommended insulin dose adjustment; and
facilitate treating the user based on the recommended insulin dose adjustment.
